(19) United States
(12) Patent Application Publication
(10) Pub. No.: US 2003/0162238 A1
(43) Pub. Date: Aug. 28, 2003

(54) Sex-chromosome-specific proteins, species specific and sperm specific proteins and methods for their identification and isolation

(75) Inventors: Stan Blecher, Guelph (CA); Jacqueline Detmar, Cambridge (CA)

Correspondence Address: FOLEY AND LARDNER, SUITE 500, 3000 K STREET NW, WASHINGTON, DC, 20007, US

(73) Assignee: University of Guelph

(21) Appl. No.: 10/365,511

(22) Filed: Feb. 13, 2003

Related U.S. Application Data
(63) Continuation of application No. 09/731,281, filed on Dec. 7, 2000, now abandoned.
(60) Provisional application No. 60/169,283, filed on Dec. 7, 1999.

(51) Int. Cl. G01N 33/53; G01N 33/537; G01N 33/543; C07H 21/04; A61K 39/395; C12P 21/02; C12N 5/06; C07K 14/47

(52) U.S. Cl. 435/7.92; 435/69.1; 435/320.1; 435/325; 424/130.1; 530/391.1; 530/350; 536/23.5

(57) ABSTRACT

Sex-chromosome-specific molecules associated with animal sperm cell membranes, and the method for isolating them, are described. An X chromosome specific molecule, a protein, having an apparent molecular weight of 32 kDa on SDS-PAGE is specifically described. The method involves preparing a cell membrane fraction from animal sperm cells; treating the cell membrane fraction with one or more substances which bind to X or Y chromosome specific molecules in the cell membrane fraction to form conjugates between the X or Y chromosome specific molecules and the substances; separating the material in the cell membrane fraction which does not bind to the substances to obtain a subfraction containing sex-chromosome-specific molecules. Methods of using the sex-chromosome-specific molecules for sperm sexing are also described.

### FIELD OF THE INVENTION

The invention relates to novel sex-chromosome-specific molecules, species specific and sperm specific proteins, more particularly X sex-chromosome-specific and Y sex-chromosome-specific proteins, methods for identifying and isolating them, and methods of their use.

### BACKGROUND OF THE INVENTION

Much research on separation of Y- and X-chromosome-bearing sperm in mammals has been conducted, to reduce the incidence of sex-lined genetic diseases or to increase animal-food production (see Windsor et al., 1993; Gledhill, 1988, and Amann, 1989). Attempts at separation have been made on the basis of various apparent differences between Y- and X-sperm, e.g. density (Harvey, 1946; Sumner and Robinson, 1976), pH sensitivity (Rothschild, 1960), swimming speed (Ericsson et al., 1973; Rhode et al., 1973), surface charge (Kaneko et al., 1984; Cartwright et al., 1993), adherence of sperm to Sephadex (Steeno et al., 1975; Adimoelja, 1987), H-Y antigen content (Goldberg et al., 1971; Peter, et al., 1993; Sills et al., 1998), motility characteristics (Sarkar, 1984; Sarkar et al, 1984) DNA content (Pinkel et al., 1982; Johnson et al., 1989) size, head shape, and mass (see review by Windsor et al., 1993, Reprod. Fert. Dev. 5:155).

Of these methods only the DNA-based technique has, to date, proven to be consistently reproducible. Artificial insertion (AI) or in vitro fertilization (IVF) result with the sorted sperm indicated that the sex ratio in cattle, rabbits and pigs were altered in the expected direction (Morrell et al., 1988; Johnson et al., 1989; Cran et al., 1993). However, even this method may not become commercially useful, due to a small yield of viable sperm and the long time required for sorting. In addition, due to the staining procedure, in which the DNA itself is labelled, and the laser-beam that the sperm are exposed to, concerns remain about the possibility of mutation and reduced long-term viability of offspring produced from such sperm. The equipment required is also large, immobile and expensive, and requires highly skilled operators.

Identification and purification of sex-specific proteins from the sperm surface have been attempted by various workers, but whether such proteins exist on the sperm surface is still in debate (Fenner et al., 1992; Cartwright et al., 1993; Hendricksen et al., 1996; Howes at al., 1997). A “male specific” antigen, the so-called H-Y (histocompatibility, Y-chromosome) on the Y-chromosome-bearing sperm has been reported in many mammalian species, e.g. cattle, goat, pig, rat and sheep (see Wachtel et al., 1988). This H-Y antigen has been used as a marker for Y- and X-chromosome-bearing sperm sorting and separation (Ali et al., 1990; Peter et al., 1993). However, other workers have reported unsuccessful Y- and X-chromosome-bearing sperm sorting using the H-Y antigen marker (Hendriksen et al., 1993; Sins et al., 1998). The consensus is that H-Y antigen cannot separate X and Y sperm.

Fabricant et al., (U.S. Pat. No. 4,722,887), describes a method for separating X- and Y-sperm by polymeric phase separation based on differential expression of a sperm cell-surface sulfoglycolipid.

Spaulding, (U.S. Pat. No. 5,021,244) describes a method for sorting sperm into enriched X- and Y-chromosome-bearing preparation. Spaulding uses DNA content and cell sorting techniques to separate the subpopulations. Spaulding only ever obtained 70-80% purity of either X or Y sperm. Johnson was able to achieve 90-95% purity. Spaulding claims to identify extracellular protein and is to provide a long string of proteins that have as yet to be identified. Spaulding also suggested that these proteins could be used to generate antibodies but in none of his work does he ever provide any data to support the existence of such antibodies. Further, the Hoechst staining and UV technique as described in U.S. Pat. No. 5,021,244 may introduce changes to the DNA. Finally, Spaulding assays that the subpopulations are enriched for each type of sperm, but Spaulding does not check that each of the separated groups of sperm are in fact X-sperm and Y-sperm. Indeed, Howes et al. (1997) and Hendricksen (1996) utilizing the methodology taught in U.S. Pat. No. 5,021,244 have failed to identify any sex-specific antigens from spermatozoa. The conclusion of their work is that an approach such as that of Spaulding's to semen sexing is unlikely to be successful.

Blecher (WO International Patent publication No. WO 97/07399) describes a method which utilizes xenogeneic immunization to produce antibodies to fetal bovine non-sex specific antigens. The antibodies to the non-sex-specific antigens are used to remove non-sex specific components of antigenic material and thus to enrich the antigenic material for residual sex-specific molecules. The sex-specific material, after purification, is then used to raise xenogeneic opposite-sex (female anti-male or male anti-female) antibodies.

### SUMMARY OF THE INVENTION

The present inventor has identified sex-chromosome-specific proteins (X-SCSPs or Y-SCSPs) which are derived from animal sperm and developed a method for identifying and isolating such sex-chromosome-specific proteins (X-SCSPs or Y-SCSPS) which are derived from animal sperm. A SCSP is a protein that is either coded for by a gene on the respective sex chromosome or coded for on another chromosome and is under the it, or indirect control of a gene on a sex-specific chromosome. As used herein, “X-chromosome specific molecules” means any molecule or epitope which is specific to or predominantly in or on X sperm as compared to Y sperm. As used herein, “Y-chromosome specific molecules” means any molecule which is specific to or predominantly in or on Y sperm as compared to X sperm. Preferably such a molecule is a protein. As used herein “X sperm” mean X sex chromosome-bearing sperm. As used herein, “Y sperm” means Y sex chromosome-bearing sperm.

The isolation of male and female sex-chromosome-specific molecules from animal sperm permits the preparation of significant quantities of antibodies with high affinity. These antibodies have utility in sexing of animal sperm cells, and will provide non-invasive methods for sexing that have both high specificity (i.e. give few false positives) and high sensitivity (give few false negatives).

Accordingly the present invention provides a method for identifying sex-chromosome-specific molecules associated with animal sperm cell membranes, comprising:

(a) infecting whole sperm, or a sperm cell fraction, from an animal of a first species (SP1) into a second and third animal where the second and third and animals are of a second species (SP2) and the SP2 animals are one of each of a male and female;

(b) harvesting antibodies raised in the second and third SP2 animals;

(c) separately reacting the antibodies from the second and third P2 animals with a sperm cell membrane fraction from a SP1 animal;

(d) separating material in the cell membrane fraction which does not bind to the antibodies from the antibodies and bound material for each of the antibodies from the second and third SP2 animals;

(e) separating the bound material from the antibodies to create bound and unbound subfractions;

(f) comparing the bound material from the second SP2 animal antibodies with the bound material from the third SP2 at antibodies and identifying as sex-chromosome-specific molecules bound material of one of the second and third SP2 animal antibodies not present in the other animal antibody bound material; and

(g) isolating the sex-chromosome-specific molecules.

Preferably the cell membrane fraction is obtained from cell membrane of bovine sperm, most preferably the cell membrane fraction is a plasma membrane fraction. Other membranes which may be used include acrosomal, mitochondrial or endoplasmic reticulum

According to another embodiment the present invention provides a purified and isolated sex-chromosome-specific molecule, preferably a protein, characterized as follows: (a) X chromosome specific; (b) associated with the cell membrane of bovine sperm cells, preferably having a molecular weight on SDS-PAGE of about 6 kDa to about 40 kDa.

In accordance with a preferred embodiment of the invention, a sex-chromosome-specific molecule, preferably a protein, is provided which is characterized as (a) X chromosome-specific; (b) associated with the plasma membrane of sperm from bovine sperm tissue; and (c) having a molecular weight of about 32 kilodaltons (kDa) as determined by sodium dodecyl sulphate-polyacrylamide gel electrophoresis (SDS-PAGE).

In accordance with a preferred embodiment, of the invention, a purified and isolated sex-chromosome-specific protein is provided having the characteristics of:

(a) X-chromosome-specific;

(b) associated with the cell membrane of a bovine sperm cell; and

(c) having a molecular weight on SDS-PAGE and pI range selected from the group consisting of: 24, 5-5.5; 23, 4.8-5.3; 21, 5.3-5.8; 20, 5.3-5.8; 14, 4.8-5.3; and 15, 5-5.5.

According to another embodiment, the present invention provides a purified and isolated, sex-chromosome-specific protein characterized as follows: (a) Y chromosome specific; (b) associated with the cell membrane of bovine sperm cells, preferably the protein having a molecular weight on SDS-PAGE of about 5 kDa to about 50 kDa, more preferably having a molecular weight range of about 10 kDa to about 25 kDa.

In accordance with a preferred embodiment of the invention there is provided a purified and isolated sex-chromosome-specific protein having the characteristics of:

(a) Y-chromosome-specific;

(b) associated with the cell membrane of a bovine sperm cell; and

having a molecular weight on SDS-PAGE and pI range selected from the group consisting of: 27, 5-6.5; 20, 5-5.5; 9, 5-5.6; 9, 53-5.8; and 5, 5.3-5.8.

According to yet another embodiment, the present invention provides a purified and isolated sex-chromosome-specific molecule, preferably a protein, characterized as follows: (a) X chromosome specific; (b) associated with the cell membrane of porcine sperm cells, preferably the protein having a molecular weight on SDS-PAGE of about 20 kDa to about 100 kDa.

In accordance with a preferred embodiment of the invention there is provided a purified and isolated sex-chromosome-specific protein characterized as follows: (a) X chromosome specific; (b) associated with the cell membrane of porcine sperm cells; and (c) having a molecular weight and pI range selected on the group consisting of 99-100, 5.3-5.7; 43, 5.3-5.7; 53, 6.1-6.7; 31, 5-5.6; 30, 6-6.5; and 25, 7.5-9.

According to yet another embodiment, the present invention provides a purified and isolated sex-chromosome-specific protein characterized as follows: (a) Y chromosome specific; (b) associated with the cell membrane of porcine sperm cells, preferably the protein having a molecular weight on SDS-PAGE of about 5 kDa to about 50 kDa.

In accordance with another preferred embodiment of the invention there is provided a purified and isolated sex-chromosome-specific protein characterized as follows: (a) Y chromosome specific; (b) associated with the cell membrane of porcine sperm cells; and (c) having a molecular weight and pI range selected on the group consisting of 36-37 and 6.2-6.8; respectively.

The sex chromosome-specific molecules identified using the methods of the invention, or isoforms or parts thereof, may be conjugated with other molecules, such as proteins, polypeptides, and/or they may be glycosylated.

The invention also permits the construction of nucleotide probes which are unique to the nuleic acid molecules encoding the sex-chromosome-specific molecules identified using the method of the invention and accordingly to the sex-chromosome-specific molecules, or isoforms, or part thereof. Thus, the invention also relates to a probe comprising a nucleotide sequence coding for a sex-chromosome-specific molecule identified using the methods of the invention. The probe may be labelled, for example, with a detectable substance and it may be used to select from a mixture of nucleotide sequences a nucleotide sequence coding for a sex-chromosome-specific molecule or parts thereof.

The molecules identified using the method of the invention, is which are isolated from tissue or recombinantly produced, may be used to prepare antibodies. The invention therefore further contemplates antibodies having specificity against an epitope of a sex-chromosome-specific molecule identified using the methods of the invention, or an isoform or part of the molecule. Antibodies may be labelled with a detectable substance and they may be used to detect a sex-chromosome-specific molecule in samples from tissues.

The sex-chromosome-specific antigens identified using the methods described herein and antibodies against an epitope of such a sex-chromosome-specific molecule, may be used to increase the probability that offspring will be of a desired sex, or that they will or will not carry a gene for a sex-chromosome linked trait.

The antibodies against an epitope of a sex-chromosome-specific molecule identified using the methods of the invention are useful for differentiating between male and female embryos, based on the determination of the presence of the sex-chromosome-specific molecule associated with a cell membrane, preferably the plasma membrane. Therefore, the invention also contemplates a method for differentiating between males and females comprising exposing an embryo or growth media of an embryo to one or more antibodies specific for an epitope of a sex-chromosome-specific molecule identified using the methods of the invention, under conditions so that a conjugate forms between the antibody and the sex-chromosome-specific molecule, and detecting the conjugates. The detection of a conjugate with antibody to a male-specific molecule determines a male, and the detection of a conjugate with antibody to a female-specific molecule deter a female.

The sex-chromosome specific molecules identified using the method of the invention may be used to identify nucleic acid molecules having sequences which encode sex-chromosome-specific molecules, preferably sex-chromosome-specific protein, more preferably a female sex-chromosome-specific protein of a molecular weight of about 32 kDa as determined on SDS-PAGE. Therefore, in accordance with an embodiment of the invention a purified and isolated nucleic acid molecule is provided containing a sequence encoding a sex-chromosome-specific molecule identified using the methods of the invention.

The nucleic acid molecules encoding sex-chromosome-specific molecules, or fragments thereof, may be inserted into an appropriate expression vector, i.e., a vector which contains the necessary elements for the transcription and translation of the inserted protein-coding sequence. Accordingly, recombinant DNA molecules adapted for transformation of a host cell may be constructed which comprise a nucleic acid molecule encoding a molecule identified using the methods of the invention, and one or more transcription and translation elements operatively linked to the nucleic acid molecule.

The recombinant molecule can be used to prepare transformed host cells expressing the molecule, or part thereof encoded by a nucleic acid molecule of the invention. Therefore, the invention further provides host cells containing a recombinant molecule of the invention.

The invention further provides a method for preparing a sex-chromosome-specific molecule, or isoforms or parts thereof utilizing the purified and isolated nucleic acid molecules identified using the methods described herein.

The invention further contemplates a method for separating male and female determining sperm from native semen which comprises treating the native sperm with one or more antibodies against (a) sex-chromosome-specific molecule(s) identified using the methods of the invention, to form conjugates between male or female-determining sperm and the antibodies, and isolating the conjugates, and sperm which have not bound to antibodies.

Antibodies against an epitope of a sex-chromosome-specific molecule identified using the methods of the invention may also be conjugated with a cytotoxin which inactivates sperm. For example, this may be achieved through antibodies which coat the sperm and the cytotoxin is then activated with photoactivation. Alternatively a magnetic bead method may be used, or agglutination. As will be readily appreciated, any other such method obvious to those skilled in the art may be used.

The invention contemplates immunizing females against X-sperm, Y-sperm, or both by administering an immunogenic amount of a sex-chromosome-specific molecule identified using the methods of the invention thereby increasing the probability of offspring of a certain sex, or decreasing fertility. Antibodies against an epitope of a sex-chromosome-specific molecule of the invention, and complement may also be used to kill X-sperm or Y-sperm in vitro or in vivo. In particular, antibodies against an epitope of a sex-chromosome-specific molecule of the invention, and complement may be placed in the reproductive tract of the female animal prior to mating to kill X- or Y-sperm.

The sex-chromosome-specific molecules identified using the methods of the invention may also be used to detect the presence of antibodies specific for the sex-chromosome-specific molecules in a sample.

The invention also relates to kits useful in performing the methods of the invention comprising antibodies against epitopes of sex-chromosome-specific molecules identified using the methods of the invention, and suitable supports useful in performing the methods of the invention.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from is detailed description.

### DESCRIPTION OF THE INVENTION

The invention will be better understood with reference to the drawings in which:

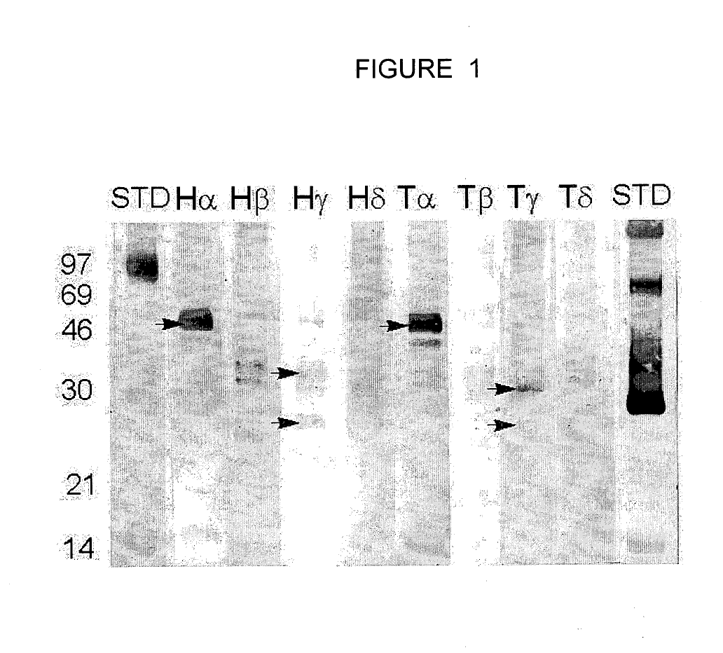

FIG. 1 is a Western blot showing sperm membrane antigens which have reacted to antibodies raised against fetal tissues.

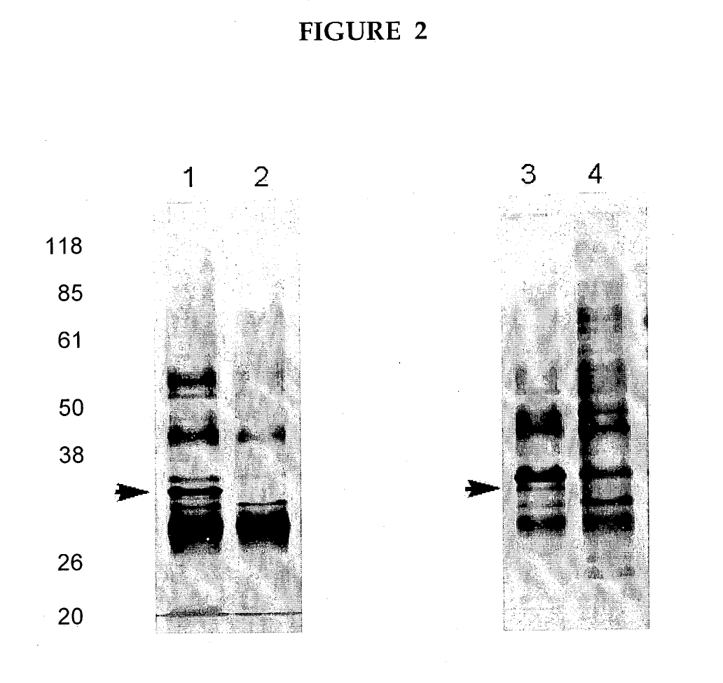

FIG. 2 is an SDS-PAGE analysis of sperm membrane fractions.

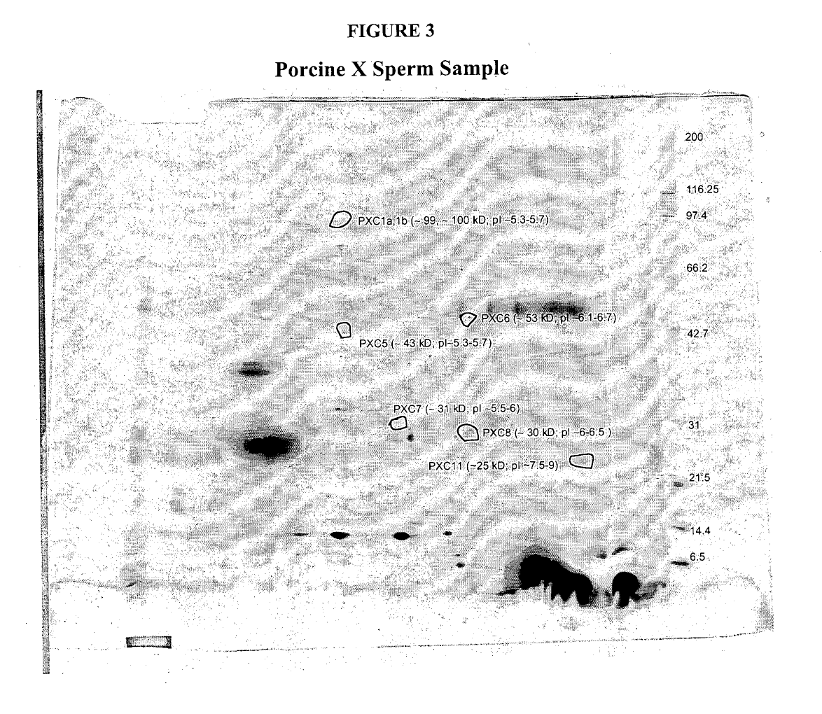

FIG. 3 is a two-dimensional polyacrylamide gel analysis of a porcine X sperm sample,

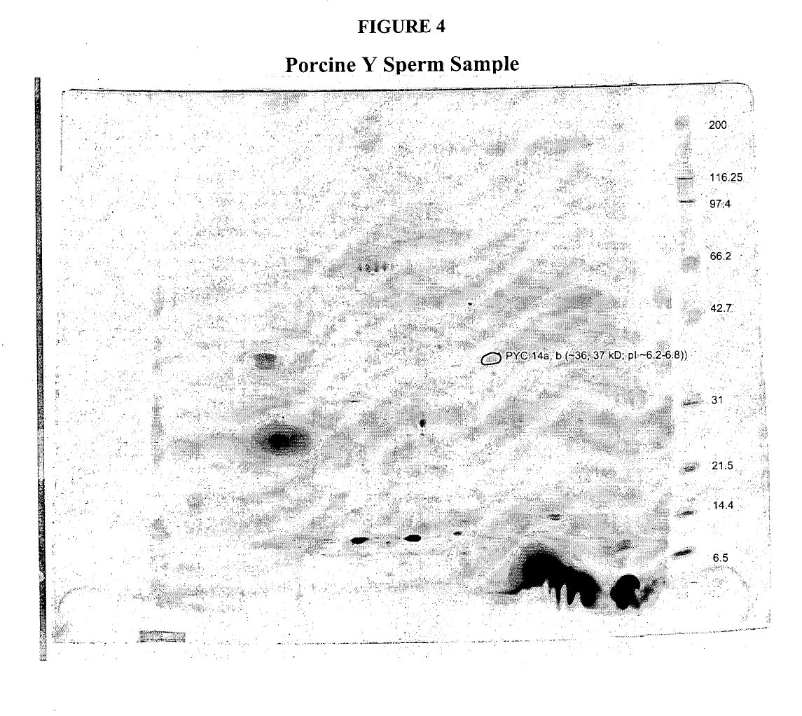

FIG. 4 is a two-dimensional polyacrylamide gel analysis of a porcine Y sperm sample.

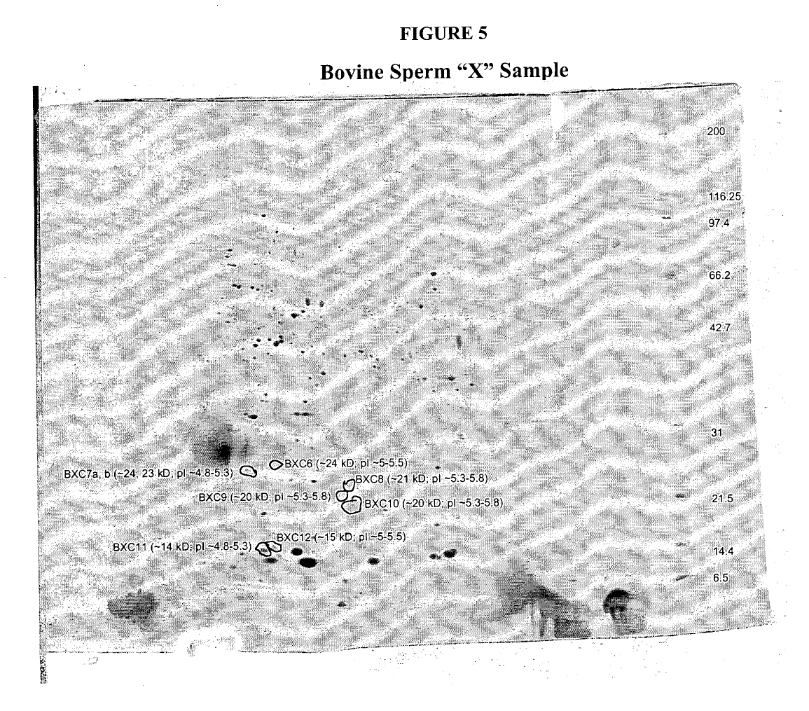

FIG. 5 is a two-dimensional polyacrylamide gel analysis of a bovine X sperm sample.

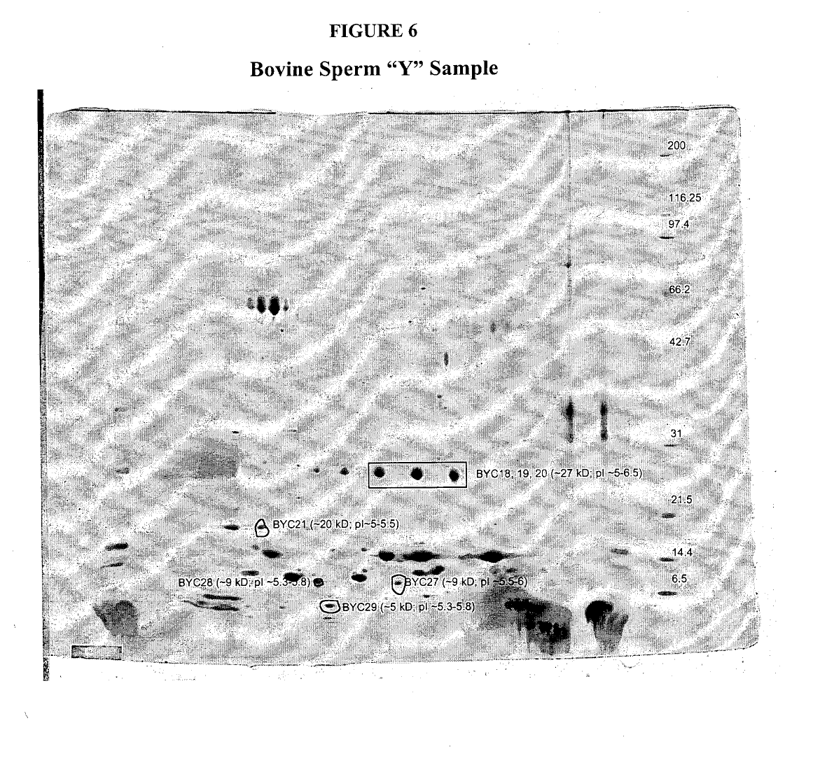

FIG. 6 is a two-dimensional polyacrylamide gel analysis of a bovine Y sperm sample.

### DETAILED DESCRIPTION OF THE INVENTION

I. Characterization of Molecules Identified Using the Method of the Invention

The present inventor has identified an X-sex-chromosome-specific molecule using the methods described herein. As illustrated in FIG. 1 and FIG. 2, a sex-chromosome-specific protein has been identified in the sperm material which is characterized as being associated with the plasma membrane of sperm cells from bovine. The molecule has a molecular weight on SDS-PAGE of about 32 kDa X-chromosome specific molecules of the present invention include those having a molecular weight, as determined on SDS-PAGE of from about 6 kDa to about 40 kDa, preferably from about 31 kDa to about 33 kDa. Y-chromosome specific molecules of the present invention include those having a molecular weight, as determined on SDS-PAGE of from about 50 kDa to about 200 kDa, preferably from about 50 kDa to about 90 kDa.

The method of the invention may be used to isolate nucleic acid molecules having sequences which encode (a) sex-chromosome-specific molecule(s). For example, the partial amino acid sequence may be determined for a sex-chromosome-specific molecule, a DNA probe may be synthesized based on the amino acid sequence, and the probe may be used to screen a cDNA library constructed from mRNA from a cell which produces the sex-chromosome-specific molecule, or a genomic DNA library. Clones containing cDNA or genomic DNA hybridizing to the probes may be isolated, and cDNA or genomic DNA sequences encoding the molecules may be identified by for example, sequencing, or by expressing the cDNA in a eukaryotic expression system and identifying clones producing protein which binds to the antibody specific to the sex-chromosome-specific molecules. The partial amino acid sequence may also be used to create primers for use in PCR to amplify the gene encoding the sex-chromosome-specific molecules. PCR-isolated genes may be sequenced and inserted into expression vectors for cloning.

Therefore, in accordance with an embodiment of the invention a purified and isolated nucleic acid molecule is provided containing a sequence encoding a sex-chromosome-specific molecule of the invention.

Fragments of the nucleic acid molecules are contemplated by the present invention. In an embodiment, the fragments include fragments that have at least 15 bases, and which are capable of hybridizing to the nucleotide sequence encoding the sex-chromosome-specific molecule under stringent hybridization conditions as described herein.

It will also be appreciated that a double stranded nucleotide sequence comprising at nucleic acid molecule of the invention or a fragment thereof, hydrogen bonded to a complementary nucleotide base sequence, and an RNA made by transcription of this nucleotide sequence are contemplated by the present invention.

Further, it will be appreciated that the invention includes other nucleic acid or amino acid sequences which have substantial sequence identity to those described herein. The term “sequences having substantial sequence identity” means those nucleic acid and no acid sequences which have slight or inconsequential sequence variations, i.e. the sequences felon in substantially the same manner to produce substantially the same polypeptides as the sequences. The variations may be attributable to local mutations, polymorphisms, or structural modifications, or the minor differences of cross-species homologies.

Stringent hybridization conditions are those which are stringent enough to provide specificity, reduce the number of mismatches and yet are sufficiently flexible to allow formation of stable hybrids at an acceptable rate. Such conditions are known to those skilled in the art and are described, for example, in Sambrook, et a., (1989, Molecular Cloning, A Laboratory Manual, Cold Spring Harbor).

The invention further provides amino acid sequences for the sex-chromosome-specific molecules of the invention and sequences which have substantial identity with the amino acid sequences. The invention still further provides peptides which are unique to the sex specific molecules of the invention. Preferably, the peptides have at least 10 to 20 amino acids, but could be as short as 5 amino acids in length.

The nucleic acid sequences contained in the nucleic acid molecules of the invention or a fragment thereof, may be inverted relative to their normal presentation for transcription to produce antisense nucleic acid molecules. The antisense nucleic acid molecules may be constructed using chemical synthesis and enzymatic ligation reactions using procedures known in the art. The antisense nucleic acid molecules of the invention or a fragment of the antisense sequence, preferably containing at least 15 bases, may be chemically synthesized using naturally occurring nucleotides or variously modified nucleotides designed to increase the biological stability of the molecules or to increase the physical stability of the duplex formed with the mRNA or the gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

II. Method of Identifying Sex-chromosome-specific Molecules

As hereinbefore mentioned, the present invention relates to a method for identifying sex-chromosome-specific molecules associated with animal sperm cells, preferably cell membranes, more preferably sperm cell plasma membranes. The molecules identified using the method of the invention, in particular sex specific molecules, include glyco-, lipo-, and phosphoproteins, polypeptides, and peptides and complexes of these molecules.

The method described herein may be applied to animals of any group within which there is sufficient evolutionary conservation of sex specific molecules, and it can accordingly be applied to a wide variety of animals. For example, it may be applied to mammals, avian species, reptiles, and fish, preferably, commercially important mammalian species including cattle, dogs, cats, horses, pigs, and sheep. It is also applicable to humans.

According to one embodiment of the method of the present invention, sex-chromosome-specific molecules associated with animal sperm cell membranes may be identified by utilizing (a) whole sperm preparation(s) from an animal. The whole sperm preparation(s) is (are) preferably unsorted, i.e., no specific enrichment of either X- or Y-sperm. In another embodiment the same procedure could be carried out on sorted sperm.

According to the method, a whole sperm preparation is obtained from a first animal, preferably cattle or pigs. However, as discussed above, the first animal may be mammal, avian, reptile or fish. The sperm preparation from a first animal is then injected into a male second SP2 animal and (a) female third SP2 animal (or vice versa). The male and female second and third SP2 animals may be litter mate siblings, and may even be inbred litter mate siblings. Again, the second and the third SP2 animals can be selected from rabbits, sheep, rats, nice, horses, cows, goats, and/or similar or corresponding approaches could be used in respect of non-mammalian species such as various fowl. However, as used herein, the second and third SP2 animals include all members of the animal kingdom. In a preferred embodiment, the SP1 animal is cattle and the second and third SP2 animals are rabbit. According to an embodiment where male and female rabbits are immunized with bovine sperm, the immunizations will result in production of male rabbit anti-bovine sperm antibodies and female rabbit anti-bovine sperm antibodies. In a preferred embodiment where SP1 and SP2 are both mammalian species, the female anti-Y sperm antibody and male anti X-sperm antibody preparations prepared by these xenogenic immunizations may be further treated to ensure that the preparations contain only antibodies to female sex-chromosome-specific molecules and male sex-chromosome-specific molecules respectively. The antibodies which bind to X or Y chromosome specific molecules in the plasma membrane of the sperm may be insolubilized to facilitate separation of a sub-fraction containing conjugates of antibodies and corresponding specific molecules. Equally untreated male rabbit anti-bovine sperm antibodies and female rabbit antibovine sperm antibodies may be insolubilized to facilitate separation of subfraction(s) containing conjugates. For example, antibodies may be bound to a suitable carrier. Examples of suitable carriers are discussed below as are methods by which insolubilization may be carried out.

According to an embodiment where untreated male and female rabbit anti-bovine sperm antibodies are used, the male rabbit anti-bovine sperm antibodies are reacted with a sperm cell membrane fraction preparation from a first animal. Separately, the female rabbit anti-bovine sperm antibodies are also reacted with a sperm cell membrane fraction from a first animal. The sperm cell membrane fraction from the first animal, may be the same animal used to provide the whole sperm preparation. However, for the purposes of this method, the sperm cell membrane fraction need not be from the same first animal, but can be from another first animal of the same species.

Bound material is separated from unbound material in respect of each of the foregoing reactions between male rabbit anti-bovine sperm antibodies and female rabbit anti-bovine sperm antibodies providing a conjugated preparation in respect of each of the male and female antibodies and an unbound fraction in respect of each preparation.

The conjugates are then treated so as to release the bound material to create a subfraction of material from each of the male and female antibodies.

Optionally, each subfraction may then be reacted with the opposite proceeding antibody, i.e., the released subfraction from the female rabbit anti-bovine sperm antibody conjugation is reacted with the male rabbit anti-bovine sperm antibodies; and the released fraction from the male rabbit anti-bovine sperm antibody preparation is reacted with the female rabbit anti-bovine sperm antibody preparation. As before, unreacted material is separated from the conjugates with subsequent release of conjugated material into two separate sub-fractions. In both cases, the sub-fractions are then subjected to an appropriate means for resolving their individual constituents such as for example, SDS-PAGE or 2-D gel electrophoresis, protein chromatography, including for example gel filtration and ion exchange chromatography. Results from the resolution of the two sub-fractions are compared and where an indication of the presence of a molecule in one means of resolution of one sub-fraction appears but does not appear in the corresponding resolution from the the sub-fraction such will be designed as a sex-chromosome-specific molecule. For example, material released from conjugates with male rabbit anti-bovine sperm antibodies is run on SDS-PAGE as is material released from female rabbit anti-bovine sperm antibodies. A band appearing in the gel from the male antibodies but not appearing in the gel where material from the female antibodies is run, or a band appearing notably more in the gel from the male antibodies and notably less in the gel where material from the female antibodies would reflect the presence of an X-chromosome specific molecule. Conversely any band appearing in material run on the gel from the female rabbit anti-bovine sperm antibody conjugations and not in the material run on the gel from the male antibodies would reflect the presence of Y-chromosome-specific molecules. Such molecules can then be isolated according to techniques described herein.

The method also involves first preparing a sperm tissue sample from an animal. The tissue sample is preferably obtained from a cell membrane, for example, the plasma membrane (outer membrane), acrosomal membrane, mitochondrial membrane, and endoplasmic reticulum membrane, most preferably the plasma membrane. For example, a plasma membrane fraction may be obtained as follows. For sperm membrane solubilization, procedures may be modified and used according to those described in Klint (1985), Fenner (1992) and Hendriksen (1995). Ejaculates from bulls for example, can be washed 3 times with HEPES-buffered saline (mass/L in ddH₂O: 8.76 g NaCl; 2.38 g HEPES, pH 7.2), pooled and centrifuged at 600×g for 10 minutes at 25° C. Triton X-100 may be added to a final concentration of 0.5% (v/v). Ten μL of 100× protease inhibitor “cocktail” (mass/mL in ddH₂O: 30.2 mg EDTA; 357 μg phenylmethinesulphonyl fluoride; 81.2 mg NEM; 811 μg Pepstatin A) should be added per mL of washed sperm. Tubes could then be shaken on ice and centrifuged at 107,000×g for 1 hour at 4° C., and protein assays performed theron. According to an alternative embodiment, membrane vesicles may be isolated using cavitation, preferably nitrogen cavitation (see for example, Gillis et al., Prep. Biochem., 8, pp. 363-378, 1978). Cell membrane vesicles consisting substantially of head cell membrane and some tail cell membrane from sperm heads, tails and other particulates may then be obtained by pelleting centrifugation, preferably centrifugation twice at 2500×g for about 30 minutes. The supernatant containing the cell membrane constituents may then be centrifuged (e.g. 100,000×g) to obtain the material to be used in the method of the invention. The material may be resuspended and washed in HEPES-buffered saline (10 mM, pH 7.2).

In an embodiment of the invention a cell membrane fraction may be obtained from sperm preparations by first preparing X and Y enriched sperm fractions. The enriched fractions may be prepared based on the DNA content of the X- and Y-sperm. According to this embodiment, the sperm preparations are subjected to flow cytometry which is based on the fact that X sperm contain more DNA than Y sperm and the X-sperm show a slightly stronger fluorescence than the Y-sperm after treatment with a DNA-binding fluorescent stain (Hoechst 33342). In another embodiment, the sperm preparations are treated with antibodies raised against male or female embryo or fetal antigens to obtain X- and Y-sperm enriched preparations.

The membrane fraction obtained is used for the preparation of an immunizing innoculent. For example, male and female SF2 animals are immunized with sperm cell membrane fractions isolated from an SP1 animal. Female SP2 thereby produces anti-Y sex-chromosome-specific SP2 antibodies and a male produces SP2 anti-X sex-chromosome-specific antibodies. The first and second animal species are selected so that an SP2 animal will produce antibodies to the sex-chromosome-specific molecules of an SP1 animal of a different sex, but are similar enough (sufficient evolutionary conservation) to not produce antibodies to sex-chromosome-specific molecules from the same sex. However, antibodies to sex specific molecules can be raised and will be raised in a third species and therefore there is no limitation with respect to choice of SP2 on the basis of evolutionary distance.

The first and second animal species may be selected from rabbits, sheep, rats, mice, horses, cattle, goats, cattle and pigs, and/or similar or corresponding approaches could be taken in respect of non-mammalian species such as various fowl. However, as used herein, animal includes all members of the animal kingdom. In an embodiment of the invention, male and female rabbits are immunized with bovine sperm cell plasma membrane fractions to produce male rabbit anti-bovine sperm antibodies and female rabbit anti-bovine sperm antibodies.

The female anti Y-sperm antibody and male anti X-sperm antibody preparations prepared by these xenogeneic immunizations may be further treated to ensure that the preparations contain only antibodies to female sex-chromosome-specific molecules and male sex-chromosome-specific molecules, respectively.

The antibodies which bind to X or Y chromosome-specific molecules in the plasma membrane fraction, or parts thereof may be insolubilized to facilitate separation of the subfraction containing the conjugates of the antibodies and X or Y chromosome specific molecules, and the subfraction containing the non-sex-chromosome-specific molecules. For example, the antibodies may be bound to a suitable carrier. Examples of suitable carriers are agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc.

The insolublized antibodies may be prepared by reacting the material with a suitable insoluble carrier using known chemical or physical methods, for example, cyanogen bromide coupling.

The conjugates of antibodies and X or Y chromosome specific molecules are isolated from the subfraction containing non-sex-chromosome-specific molecules by conventional isolation techniques, for example, salting out, chromatography, electrophoresis, gel filtration, fractionation, absorption, polyacrylamide gel electrophoresis, agglutination, or combinations thereof. When the antibody is insolubilized the conjugates may be eluted using conventional procedures.

In an embodiment of the invention, the sex-chromosome-specific molecules are isolated by molecular size and/or pI, using techniques known in the art. Electrophoresis according to standard practice as described in Sambrook, J. et al. (Molecular Cloning A Laboratory Manual Cold Spring Harbour Laboratory Press, Sections 6.3-6.9, 1989 which is incorporated herein by reference) may be used to separate the sex-chromosome-specific molecules, and supports such as gel sheets or slabs, for example, polyacrylamide, agarose or other polymers are typically used as the supporting medium. Preferably, two dimensional gels which separate the proteins on the basis of two characteristics e.g. molecular size and pI are employed; most preferably SDS-polyacrylamide gel electrophoresis (SDS/PAGE), or immobilized pH gradient gel SDS-polyacrylamide gel electrophoresis (IPG-SDS/PAGE) are used to separate the sex-chromosome-specific molecules. The sex-chromosome-specific molecules may be eluted or removed from the gels using conventional procedures such as described by Lee et al. (1987, Analyt. Biochem. 166:308).

III. Preparation of Molecules Identified Using the Method of the Invention

Nucleic acid molecules encoding the sex-chromosome-specific molecules identified using the method of the invention, or fragments thereof, may be isolated and sequenced using the procedures described above or they may be constructed by chemical synthesis and enzymatic ligation reactions using procedures known in the art.

The sex-chromosome-specific molecules of the invention, or isoforms or parts thereof, may be prepared using recombinant DNA methods. Accordingly, the nucleic acid molecules having a sequence which codes for a sex-chromosome-specific molecule or fragments thereof may be incorporated in a known manner into an appropriate expression vector which ensures good expression of the molecules, or isoforms, or parts thereof. Possible expression vectors include but are not limited to cosmids, plasmids, or modified viruses, so long as the vector is compatible with the host cell used.

The invention therefore contemplates a recombinant molecule containing a nucleic acid molecule encoding a sex-chromosome-specific molecule identified using the method of the invention, or fragments thereof, and the necessary elements for the transcription and translation of the inserted sequence. Suitable transcription and translation elements may be derived from a variety of sources, including bacterial, fungal, viral, mammalian, or insect genes. Selection of appropriate transcription and translation elements is dependent on the host cell chosen, and may be readily accomplished by one of ordinary skill in the art. Examples of such elements include: a transcriptional promoter and enhancer or RNA polymerase binding sequence, a ribosomal binding sequence, including a translation initiation signal. Additionally, depending on the host cell chosen and the vector employed, other genetic elements, such as an origin of replication, additional DNA restriction sites, enhancers, and sequences conferring inducibility of transcription may be incorporated into the expression vector. It will also be appreciated that the necessary transcription and translation elements may be supplied by the native gene and/or its flanking regions.

The recombinant molecules may also contain a reporter gene which facilitates the selection of host cells transformed or transfected with a recombinant molecule of the invention. Examples of reporter genes are genes encoding proteins such as β-galactosidase, chloramphenicol acetyltransferase, and firefly luciferase. Transcription of the reporter gene is monitored by changes in the concentration of the reporter protein such as β-galactosidase, chloramphenicol acetyltransferase, or firefly luciferase. This makes it possible to visualize and assay for expression of recombinant molecules.

Recombinant molecules can be introduced into host cells by transformation, transfection, infection, electroporation etc. Methods for transforming transfecting, etc. host cells to express foreign DNA are well known in the art (see, e.g., Itakura et al., U.S. Pat. No. 4,704,362; Hinnen et al., PNAS USA 75:1929-1933, 1978; Murray et al., U.S. Pat. No. 4,801,542; Upshall et al., U.S. Pat. No. 4,935,349; Hagen et al., U.S. Pat. No. 4,784,950; Axel et al., U.S. Pat. No. 4,399,216; Goeddel et al., U.S. Pat. No. 4,766,075; and Sambrook et al. Molecular Cloning A Laboratory Manual 2nd edition, Cold Spring Harbor Laboratory Press, 1989, all of which are incorporated herein by reference).

Suitable host cells include a wide variety of prokaryotic and eukayotic host cells, including bacterial, mammalian, yeast or other fungi, viral, plant, or insect cells.

The sex-chromosome-specific molecules or isoforms or parts thereof may also be prepared by chemical synthesis using techniques well known in the chemistry of proteins such as solid phase synthesis (Merrifield, 1964, J. Am. Chem. Assoc. 85;2149-2154) or synthesis in homogeneous solution (Houbenweyl, 1987, Methods of Organic Chemistry, ed. E. Wansch, Vol. 15 I and II, Thieme, Stuttgart).

The sex-chromosome-specific molecules of the invention, or isoforms or parts thereof, may be conjugated with other molecules, such as proteins or polypeptides. Fusion proteins may be prepared by fusing, through recombinant techniques, a region of the sex-chromosome-specific molecules or parts thereof, and a selected protein or marker protein with a desired biological function. Examples of proteins which may be used to prepare fusion proteins include cytotoxins and immunogenic protein. They may also be conjugated to other specific molecules, including antibodies, to direct localization of the molecules to specific target sites. In addition the genes coding for the molecules may be inserted into expression vectors under the control of site-specific promoters, to target specific sites. Genetic constructs may also be made containing coding sequences for sex specific molecules and sequences for strongly immunogenic molecules, in order to improve the antigenicity of the molecules (Tao and Levy, 1993, Nature Vol. 362:755-758).

The preset invention also contemplates a method for screening for epitopes of sex-chromosome-specific molecules tat are presented by Major Histocompatibility Complex molecules. This may be accomplished by isolating plasma membrane preparations of macrophage or monocyte cells that have been pulsed with a sex-chromosome-specific molecule, and isolating the epitopes using the antibodies specific for the sex-chromosome-specific molecules obtained by the methods described herein.

IV. Applications for the Molecules Identified Using the Method of the Invention

The nucleic acid molecules encoding the sex-chromosome-specific molecules identified using the method of the invention, or fragments thereof, allow those skilled in the art to construct nucleotide probes for use in the detection of nucleotide sequences in biological materials. A nucleotide probe may be labelled with a detectable substance such as a radioactive label which provides for an adequate signal and has sufficient half-life such as ³²P, ³H, ¹⁴C or the like. Other detectable substances which may be used include antigens that are recognized by a specific labelled antibody, fluorescent compounds, enzymes such as lac Z, antibodies specific for a labelled antigen, and chemiluminescense. An appropriate label may be selected having regard to the rate of hybridization and binding of the probe to the nucleotide sequence to be detected and the amount of nucleotide available for hybridization. Labelled probes may be hybridized to nucleic acids on solid supports such as nitrocellulose filters or nylon membranes as generally described in Sambrook et al., 1989, Molecular Cloning, A Laboratory Manual (2nd ed.). The nucleotide probes may be used to detect genes that encode the sex-chromosome-specific molecules identified using the methods of the invention.

The sex-chromosome-specific molecules identified using the methods of the invention, or isoforms and parts thereof, may be used to prepare antibodies. Antibodies having specificity for the molecules may also be raised against proteins prepared by expressing nucleic acid molecules encoding the molecules in a host cell as described above.

Within the context of the present invention, antibodies are understood to include monoclonal antibodies, polyclonal antibodies, antibody fragments (e.g., Fab, and F(ab′)₂ and recombinantly produced binding partners.

Polyclonal antibodies may be readily generated by one of ordinary skill in the art from a variety of warm-blooded animals such as horses, cattle, pig, various fowl, rabbits, goat, sheep, rabbits, mice, or rats. Briefly, a sex-chromosome-specific molecule is utilized to immunize the animal though intraperitoneal, intramuscular, intraocular, intravenous, subcutaneous, intranondal, intrasplenic implantation e.g. using nitrocellulose as a carrier, or subcutaneous injections, in conjunction with an adjuvant such as Freund's complete or incomplete adjuvant, or following conjugation or chemical modification to increase antigenicity. Following several booster immunizations, samples of serum are collected and tested for reactivity to the sex-chromosome-specific molecule. Particularly preferred polyclonal antisera will give a signal on one of these assays that is at least three times greater than background. Once the titre of the animal has reached a plateau in terms of its reactivity to the sex-chromosome-specific molecule, larger quantities of antisera may be readily obtained either by weekly bleedings, or by exsanguinating the animal.

Monoclonal antibodies may also be readily generated using conventional techniques (see Kohler and Milstein, Nature 256, 495-497, 1975 which is incorporated herein by reference; see also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference).

Briefly, within one embodiment a subject animal such as a rat or mouse is injected with a sex-chromosome-specific molecule. The molecule may be mixed with an adjuvant such as Freund's complete or incomplete adjuvant in order to increase the resultant immune response. Between one and three weeks after the initial immunization the animal may be reimmunized with another booster immunization, and tested for reactivity to the molecule. Once the animal has plateaued in its reactivity to the protein, it is sacrificed, and organs which contain large numbers of B cells such as the spleen and lymph nodes are harvested.

Cells which are obtained from the immunized may be immortalized by transfection with a virus such as the Epstein Barr virus (EBV) (see Glasky and Reading, Hybridoma 8(4):377-389, 1989). Alternatively, the harvested spleen and/or lymph node cell suspensions are fused with a suitable myeloma cell in order to create a “hybridoma” which secretes monoclonal antibody. Suitable myeloma lines include, for example, NS-1 (ATCC No. TIB 18), and P3X63-Ag 8.653 (ATCC No. CRL 1580).

Following the fusion, the cells may be placed into culture plates containing a suitable medium, such as RPMI 1640, or DMEM (Dulbecco's Modified Eagles Medium) (JRH Biosciences, Lenexa, Kans.), as well as additional ingredients, such as Fetal Bovine Serum (FBS, e.g., from Hyclone, Logan, Utah, or JRH Biosciences). Additionally, the medium should contain a reagent which selectively allows for the growth of fused spleen and myeloma cells such as HAT (hypoxante aminopterin, and thymidine) (Sigma Chemical Co, St. Louis, Mo.). After about seven days, the resulting fused cells or hybridomas may be screened in order to determine the presence of antibodies which are reactive against the sex-chromosome-specific molecule. A wide variety of assays may be utilized to determine the presence of antibodies which are reactive against a sex-chromosome-specific molecule, including for example, fluorescence activated cell sorting, countercurrent immunoelectrophoresis, radioimmunoassays, radioimmuno-precipitations, enzyme-linked immuno-sorbent assays (ELISA), dot blot assays, inhibition or competition assays, and sandwich assays (see U.S. Pat. Nos. 4,376,110 and 4,186,530; see also Antibodies: A Laboratory Manual, Harlow and Lane (eds.), Cold Spring Harbor Laboratory Press, 1988). Following several dilutions and reassays, a hybridoma producing antibodies reactive against a sex-chromosome-specific molecule may be isolated.

Other techniques may also be utilized to construct monoclonal antibodies (see William D. Huse et al., “Generation of a Large Combinational Library of the Immunoglobulin Repertoire in Phage Lambda,” Science 246:1275-1281, December 1989; see also L. Sastry et al, “Cloning of the Immunological Repertoire in Escherichia coli for Generation of Monoclonal Catalytic Antibodies: Construction of a Heavy Chain Variable Region-Specific cDNA Library;” Proc Natl. Acad. Sci USA 86:5728-5732, August 1989; see also Michelle Alting-Mees et al., “Monoclonal Antibody Expression Libraries: A Rapid Alternative to Hybridomas,” Strategies in Molecular Biology 3:1-9, January 1990; these references describe a commercial system available from Stratacyte, La Jolla, Calif., which enables the production of antibodies through recombinant techniques).

Similarly, binding partners may also be constructed utilizing recombinant DNA techniques to incorporate the variable region of one gene and the constant region of another gene e.g. a non-human animal variable region and a human constant region. Within one embodiment, the genes which encode the variable region from a hybridoma prodrug a monoclonal antibody of interest are amplified using nucleotide primers for the variable region. These primers may be synthesized by one of ordinary skill in the art, or may be purchased from commercially available sources. Primers for mouse and human variable regions are available from Stratacyte (La Jolla, Calif.). These primers may be utilized to amplify heavy or light chain variable regions, which may then be inserted into vectors such as ImmunoZAP™ H or ImmunoZAP™ L. (Stratacyte), respectively. These vectors may then be introduced into E. coli for expression. Utilizing these techniques, large amounts of a single-chain protein containing a fusion of the V_{H} and V_{L} domains may be produced (See Bird et al., Science 242:423-426, 1988). In addition, such techniques may be utilized to change a “murine” antibody to a “human” antibody, without altering the binding specificity of the antibody.

Once suitable antibodies or binding partners have been obtained, they may be isolated or purified by many techniques well known to those of ordinary skill in the art (see Antibodies: A Laboratory Manual, Harlow and Lane (eds.), Cold Spring Harbor Laboratory Press, 1988). Suitable techniques include peptide or protein affinity columns, HPLC or RP-HPLC, purification on protein A or protein G columns, or any combination of these techniques.

The specificity of antibodies or binding partners for sex-chromosome-specific molecules may be confirmed by reacting with purified antigen preparations. For example, the specificity of antibodies for female sex-chromosome-specific antigens may be confirmed by reacting the antibodies with a tissue sample prepared from a parthenogenote which is free of male sex-chromosome-specific antigens.

In one embodiment of the invention, antibodies to male- or female-sex-chromosome-specific molecules associated with bovine cell membranes are raised by injecting the purified bovine sex-chromosome-specific molecules into an appropriate recipient animal of a different species, preferably rabbits, sheep and goats. Each band in the 1-dimensional gel electrophoretogram shown in FIG. 1 likely represents more than one protein, as demonstrated by 2-dimensional electrophoresis. The antibodies produced by each band are therefore oligospecific; that is, they have reactivity to a small number (e.g. three or four) different antigens. By using 2-dimensional electrophoresis, single molecules are preferentially isolated and monospecific antibodies produced. In particular, using 2-dimensional Western blotting, the most antigenic, sex specific, and avidly binding molecules are identified.

The polyclonal or monoclonal antibodies to sex-chromosome-specific molecules may be used to purify the sex-chromosome-specific antigens and to detect sex-chromosome-specific molecules, or isoforms or parts thereof, in embryos, various cells and tissues (e.g. sperm cells, spleen, kidney, ovary, and testes, extracts and cells), and biological materials (e.g. body fluids such as blood, urine, and blastocoelic fluid, and amniotic fluids). The antibodies may also be used to quantify the amount of a sex-chromosome-specific molecule, or an isoform or part thereof, in a sample in order to determine its role in particular cellular events or pathological states. In particular, the polyclonal and monoclonal antibodies of the invention may be used in immuno-histochemical analyses, for example, at the cellular and sub-subcellular level, to detect a sex-chromosome-specific molecule of the invention, to localise it to particular cells, tissues, embryos, and organisms and to specific subcellular locations, and to quantitate the level of expression.

The antibodies may also be used to detect cells from a particular species in tissue culture and in hybridoma studies.

Direct methods may be employed in which the antibody is labelled with a detectable substance as described above. Indirect methods may also be employed in which the primary antigen-antibody reaction is amplified by the introduction of a second antibody, having specificity for the antibody reactive against the sex-chromosome-specific antibody. By way of example, if the antibody having specificity against the sex-chromosome-specific molecule of the invention is a rabbit IgG antibody, the second antibody may be goat anti-rabbit immunoglobulin G labelled with a detectable substance as described herein. Generally, an antibody of the invention may be labelled with a detectable substance and the sex-chromosome-specific molecules of the invention may be detected based upon the presence of the detectable substance. Examples of detectable substances include various enzymes, fluorescent materials, luminescent materials, biotin, magnetic particles, micro- or macro-particles, and radioactive materials. Examples of suitable enzymes include horseradish peroxidase, alkaline phosphatase, β-galactosidase, or acetylcholinesterase; examples of suitable fluorescent materials include umbelliferone, fluorscein, fluorescein isothiocyanate, rhodamine, dichlorotriazinylamine fluorescein, dansyl chloride or phycoerythrin; an example of a luminescent material includes luminol; and examples of suitable radioactive material include radioactive iodine I¹²⁵, I¹³¹ or tritium. Antibodies may also be coupled to electron dense substances, such as ferritin or colloidal gold, which are readily visualised by electron microscopy.

Radioactive labelled materials may be prepared by radiolabeling with ¹²⁵I by the chloramine-T method (Greenwood et al., Biochem. J. 89:114, 1963), the lactoperoxidase method (Marchalonis et al., Biochem. J. 124:921, 1971), the Bolton-Hunter method (Bolton and Hunter, Biochem. J. 133:529, 1973 and Bolton Review 18, Amersham International Limited, Buckinghamshire, England, 1977), the iodogen method (Fraker and Speck, Biochem. Biophys. Res. Commun. 80:849, 1978), the Iodo-beads method (Markwell Anal. Biochem. 125:427, 1982) or with tritium by reductive methylation (Tack et al., J. Biol. Chem. 255:8842, 1980).

Known coupling methods (for example Wilson and Nakane, in “Immunofluoroescence and Related Staining Techniques”, W. Knapp et al., eds, p. 215, Elsevier/North-Holland, Amsterdam & New York, 1978; P. Tijssen and E. Kurstak, Anal. Biochem. 136:451, 1984) may be used to prepare enzyme labelled materials. Fluorescent labelled materials may be prepared by reacting the material with umbelliferone, fluorescein, fluorescein isothiocyanate, dichlorotriazinylamine fluoroescein, dansyl chloride, derivatives of rhodamine such as tetramethyl rhodamine isothiocyanate, or phycoerythrin.

When labelled antibody is used, the sex-chromosome-specific molecules can be detected by measuring the labelled antibody-antigen conjugates. The appropriate method of measuring the labelled conjugates is dependent upon the detectable substance employed. For example, if the labelling agent is an enzyme, the sex-chromosome-specific molecule may be detected using a proper enzyme substrate for colorimetric, luminescent or fluorescent systems. If the labelling agent is a fluorescent material, the presence of a sex-chromosome-specific molecule may be determined by fluorescence intensity, and if the labelling agent is a radioactive material, the sex-chromosome-specific molecule of the invention may be localized by radioautography. The results of radioautography may be quantitated by determining the density of particles in the radioautographs by various optical methods, or by counting the grains.

The antibody against a sex-chromosome-specific molecule may be insolubilized by binding to a suitable carrier. Examples of suitable carriers are described herein. The insolubilized antibody may be prepared by reacting the material with a suitable insoluble carrier using known chemical or physical methods, for example, cyanogen bromide coupling.

The antibodies and antibodies labelled with a detectable substance as discussed above, may be used to detect the presence of sex-chromosome-specific molecules in conventional assays such as ELISA, radioimmunoassays, inhibition or competition assays, sandwich assays, Dot Blot assays, Radioimmunoprecipitation, or histochemical tests.

By way of example, the antibodies may be used to detect a sex-chromosome-specific molecule in a cell, tissue or biological material in an inhibition assay in which extracts of the material to be tested are coated to a plate, the antibody is reacted with increasing amounts of antigen in a test solution, and the presence of antigen in the test solution is quantified in relation to the amount of inhibition occurring when the pretreated antibody is allowed to react with the coated antigen in the plate. In another sandwich method or capture assay, purified antibody against a sex-chromosome-specific molecule is bound to a plate, varying amounts of a putative source of antigen are introduced, the plate is washed and the amount of bound antigen is determined by use of biotin-conjugated antibody and avidin-biotinylated peroxidase indicator.

The antibodies and nucleic acid probes suitable for detecting sex-chromosome-specific molecules may be packaged into convenient kits providing the necessary materials packaged into suitable containers. For example, such kits may include a series of antibodies against sex specific molecules. The kits may also include suitable supports useful in performing the methods of the invention.

The antibodies, nucleic acid probes and kits of the present invention have many practical applications. Sex-chromosome specific molecules identified using the methods described herein are present on the cellular membranes of cells. By exposing embryos to specific antibodies to these sex-chromosome-specific molecules, it is possible to identify the sex of the embryos, for example using detectable substances that can be bound to the antibodies. Embryos, selected in this way can be recovered, the antibody can be washed off, continued in vitro culture followed by transfer to cows can be done, and successful gestation can result.

Thus, the invention also broadly contemplates a method for differentiating between males and females comprising exposing an embryo or growth media of an embryo, to one or more antibodies specific for an epitope of a sex-chromosome-specific molecule identified using the methods of the invention, under conditions so that a conjugate forms between the antibodies and the sex-chromosome-specific molecule, and detecting the conjugates. The detection of a conjugate with antibody to a chromosome specific molecule determines a male, and the detection of a conjugate with antibody to an X chromosome specific molecule determines a female. Anti-X chromosome and anti-Y chromosome specific molecule antibodies may be used separately, in combination, or sequentially to differentiate between males and females. The direct and indirect methods discussed above which are embodied in conventional assays such as ELISA, radioimmunoassays, or histochemical tests may be used to sex embryos.

Embryos which may be sexed using the methods described herein may be obtained from mammalian species including, cattle, dogs, cats, horses, swine, goats, and sheep. Similar or corresponding approaches could be taken in non-mammalian animals including avian species, fish, and reps. In some of these animals, the females are heterogametic (whereas in males are heterogametic) and therefore, some mammalian male sex-chromosome-specific molecules may be homologous to female sex-chromosome-specific molecules of some of these non-mammalian species, and vice versa.

The embryos may be in vitro or in vivo fertilized embryos, or parthenogenotes.

Embryos may be obtained using conventional techniques. For, example, embryos may be obtained from superovulated sheep, goats, pigs and cattle. Sheep, goats, pigs, and cattle may be injected with FSH-P descending divided doses at 12 hour intervals for about 3 days, followed by injection of a prostaglandin analogue (e.g. Ono-1052, Ono Pharma. Co. Ltd., Japan). Embryos may be collected laparoscopically from goats and sheep. Bovine embryos may be collected nonsurgically by flushing the uteri of superovulated donors at about 6 to 7 days after estrus and artificial insemination. The embryos may be cultured at 37° C. in 5% CO₂: 95% air for about six hours in 10% bCS-supplemented BMOC-3 media (Brinster, RL, 1972: Cultivation of the mammalian embryo. In: G. Rothblat, V J Cristfalo (eds); “Nutrition and Metabolism of Cells in Culture,” Vol, 2. New York: Academic Press, pp. 252-286).

Antibody specific for an epitope of a sex-chromosome-specific molecule identified using the methods of the invention, may be prepared using the methods descried herein. The conditions which may be employed so that a conjugate forms between the antibody and the sex-chromosome-specific molecule are generally known in the art. The amount of antibody used to form the conjugate may be selected based on the type of antibody, and the properties of the sex-chromosome-specific molecule. The conjugates may be separated by conventional isolation techniques, for example, salting out, chromatography, electrophoresis, gel filtration, fractionation, absorption, polyacrylamide gel electrophoresis, agglutination, or combinations thereof.

The following three techniques or combinations thereof are preferably used for separating embryos:

a) A double antibody method. The embryos may be exposed to antibodies against one or more sex-chromosome-specific molecules, followed by fluorescein-labelled anti-gammaglobulin second antibody. Sequential use of anti-male and anti-female antibodies, followed by their respective antibodies may be used. This method allows for manual separation of labelled from unlabelled embryos.

b) The embryos may be separated based on their morphology when incubated with an antibody to a sex-chromosome-specific molecule, comparable to the procedures set out in Utsumi et al., (1993, Mol. Reprod. Devel. 36:238). The antibodies may reversibly retard the growth of male but not female embryos when using anti-male antibody or vice versa when using anti-female antibody. The antibodies may also be used with or without additives, e.g. complement, to irreversibly suppress or to kill embryos of one sex, leaving substantially pure cults of the other sex. This method also allows for manual separation.

c) Magnetic bead labelling. In this method, embryos are exposed to commercially available, microscopically small magnetic beads coated with appropriate antibodies (e.g. Olsaker et al., 1993, Anim. Genet. 24:311), in this case either male-specific or female-specific antibodies. Magnetic beads coated with commercially available goat anti-rabbit immunoglobulin may be added to embryos previously exposed to male specific or female specific antibodies. Alternatively, the beads may be coated, for example with anti-rabbit immunoglobulin and then with male-specific antibody, and placed directly in a suspension of embryos, in an appropriate receptacle. Because the sex-specific proteins are present on the epithelial cell surface, male embryos will bind to the male specific antibody on the beads, while female embryos will not. The beads and attached embryos are pulled to the side of the dish, using a magnet. Commercially available combinations of second antibody and avidin-biotin enhanced magnetic beads may also be used, for example, Protein A or Protein G coated magnetic beads.

The sex of the separated embryos may be confirmed using known procedures such as chromosomal analysis and/or by DNA methods.

Sperm cell membranes contain molecules that react with Y chromosome-specific molecule or X chromosome-specific molecule antibodies produced using the methods of the invention. The different molecules, male-chromosome specific and female-chromosome specific, are situated in the two classes of sperm, Y and X respectively.

Therefore, the present invention also contemplates a method for separating male and female determining sperm from native sperm which comprises incubating the native sperm with one or more antibodies against a sex-chromosome-specific molecule identified using the methods of the invention, to form conjugates between male or female determining sperm and the antibodies, and isolating the conjugates and sperm which have not bound to conjugates. The antibodies used in the method are antibodies against male- and female-chromosome specific molecules isolated from sperm cell plasma membrane preparations.

The antibodies against X- or Y-chromosome specific antigens may bind to and inactivate X- or Y-sperm respectively, and may, under certain circumstances, prevent them from fertilizing an ovum. The sperm cells not bound by the antibodies may remain viable and active for fertilizing ova. Thus, the invention provides a method to produce a semen sample enriched in active X- or Y-sperm and thus capable of increasing the probability that offspring will be of a desired sex, or will or will not carry a gene for a sex-chromosome linked trait.

The magnetic bead method (e.g. as described by Olsaker et al., 1993, supra) may be used to separate putative X- and Y-sperm. The beads, coated, for example with male-chromosome specific antibody, may be placed in a suspension of the sperm-cells, in an appropriate receptacle. Because the sex-chromosome specific proteins are present in the sperm cell plasma membranes, the Y-sperm cells bind to the male chromosome specific molecule antibody on the beads, while the X-sperm will not. The beads are then pulled to the side of the dish, using a magnet. Sperm cells of the two classes, those adhering to the beads (Y) and those not adhering (X), are recovered.

The following method may also be used to separate male and female determining spermatozoa. A native sperm preparation may be exposed to a first antibody that binds male chromosome specific molecules. The exposed sperm may be suspended together with a conjugate of a second antibody that binds exclusively to the first antibody and an immunoabsorbent substrate in a protein-free diluent to form a conjugate/sperm preparation whereby the producing or Y chromosome-bearing sperm are bound to the substrate. The sperm may then be recovered from the substrate by specific binding of the substrate.

The methods do not require mechanical handling; are non-invasive; they do not require chemical binding to cellular internal structures; they involve minimal manipulation; they are inexpensive; there are minimal requirements for equipment or instrumentation; and, they are readily and easily done.

The antibodies against sex-chromosome-specific molecules identified using the methods of the invention may also be used to control the sex of progeny in vivo. For example, females may be immunized against X-sperm, Y-sperm or both using vaccines containing the sex-chromosome-specific antigens identified using the methods of the invention thereby increasing the probability of offspring of a certain sex or decreasing fertility altogether. The sex of an animal's (preferably mammal's) progeny may be controlled to produce more females or males by placing antibody against X or Y chromosome-specific molecules respectively, and complement in the uterus or reproductive tract prior to coitus to affect/select sperm to kill the non-required sperm.

Antibodies against an epitope of a sex-chromosome-specific molecule identified using the methods of the invention may also be conjugated with a cytotoxin which inactivates sperm. Thus, the cytotoxin may be specifically targeted to sperm. These preparations may therefore be useful as a contraceptive. Antibodies to the male and female specific molecules identified using the methods described herein may also be useful as a contraceptive by contacting sperm with both the anti-male and anti-female antibodies. Antisense sequences to the male and female specific molecules may also have utility as contraceptives.

The sex-chromosome-specific molecules identified using the methods of the invention may also be used to detect the presence of antibodies specific for the sex-chromosome-specific molecules in a sample.

The antibodies specific for sex-chromosome-specific molecules identified using the methods of the invention are also important in the medical field for prevention of lethal sex linked genetic diseases in humans. For example, X or Y chromosome specific antibodies may be used to produce a semen sample enriched in active Y-sperm thus increasing the probability that offspring will not carry a gene for a sex-chromosome linked trait.

The following non-limiting examples are illustrative of the present invention:

### EXAMPLES

### Background Procedure to Examples

### Preparation of Plasma Membrane Protein From Sperm

Fresh sperm and cryopreserved sperm (in egg yolk extender) were obtained from GenCor, Guelph. For Western blots, the nitrogen cavitation procedure was used (Buhr, M. M. et al. (1994); and Buhr, M. M. personal communication). For sperm membrane solubilization, procedures were modified from Klint (1985); Fenner (1992) and Hendriksen (1995). Ejaculates from three bulls were washed 3 times with HEPES-buffered saline (mass/L in ddH₂O: 8.76 g NaCl; 2.39 g HEPFS, pH 7.2), pooled and centrifuged at 600×g for 10 minutes at 25° C. Triton X-100 was added to a final concentration of 0.5% (v/v). Ten μL of 100× protease inhibitor “cocktail” (mass/mL in ddH₂O: 30.2 mg EDTA; 357 μg phenylmethanesulphonyl fluoride; 81.2 mg NEM; 811 μg Pepstatin A) was added per mL of washed sperm. Tubes were shaken on ice for 1 hour, centrifuged at 107,000×g for 1 hour at 4° C., and protein assays performed.

### Immunoblotting (Western Blotting)

Western blotting was done as described in Sambrook (Molecular Cloning: A Laboratory Manual, 2nd edition, Vol. 3, pp. 18.60-18.75, 1989). Bound antibodies were detected using protein A-horseradish peroxidase, and diaminobenzidine as substrate. All primary antibodies used for immunoblotting were previously analyzed by ELISA (Hudson, 1980) to establish titre. Female and male antigens were coated at 1 ug/well (for unpurified solubilized proteins) or 100 ng/well (for semi-purified or purified SSP).

### Electrophoresis

Protein fractions were resolved by SDS-PAGE, using the standard Mini-Gel procedure (BioRad) as described in the manufacture's instructions. Gels were stained in 0.2% a (wt/vol) Coomassie Blue or with silver stain.

### Antibodies to Fetal Membrane Proteins

New Zealand White rabbits, 3.0-3.5 kg, (Maple Lane Rabbitry and Charles River Rabbitry, Charles River, Canada), were immunized by 4 subcutaneous (sc) injections followed by an intramuscular (im) boost. The antisera were assigned Greek letter symbols as follows: female rabbit anti-male tissue—alpha (α); femal rabbit anti-female bovine—beta (β); male rabbit anti-femal bovine—gamma (γ); male rabbit anti-male bovine—delta (δ).

### Antibodies to Sperm Membrane Protein

Male and female rabbits were immunized with pooled, fresh or cryopreserved sperm (4×subcutaneous (sc) and 1 intramuscular (im)). Sperm cells were washed twice in HEPES-buffered saline (HBS) Sperm TALP (Tyrodes, Albumin, lactate, pyruvate) (Rieger, E. et al. J. Reprod. Fertil. 1995; 105:918), with centrifugation at 200×g for 10 minutes at room temperature (RT). This relatively non-stringent treatment was used in order to preserve cellular viability Sperm counts were done in hemacytometer. Each sc injection contained 39.9×10⁶ pooled sperm+200 μL Freund's incomplete adjuvant (FIA); the im boost contained 79.8×10⁶ pooled sperm+200 μL FIA (modified from Ambrose (J. Androl. 1996; 17:567-578;), Castle (Biol. Reprod. 1997; 56:153-159) and Howes (1997)). The antisperm antisera from female and male rabbits were assigned the Greek letter symbols epsilon (ε) and zeta (ζ) respectively. These are putative “anti-Y” and “anti-X” antisera respectively. To test the specificity of these antisera for sperm membrane protein, immunocytochemistry was performed on methanol-fixed smears of cryopreserved sperm. Second antibody was Fluorosceinisothiocyanate (FITC)-conjugated goat anti-rabbit IgG (Sigma Chemical Co.)(1/10 in ddH₂O). The slides were viewed under a Zeiss™ IM35 UV microscope.

### Fetal Membrane Protein Purification

Preclearance of solubilized fetal plasma membrane proteins was done using CNBr-activated Sepharose 4B affinity columns (Amersham Pharmacia Biotech) with rabbit preimmune sera as ligand. Unbound and bound eluted fractions were collected. The fractions were analyzed by absorbance (A280), protein assay (Pierce), SDS-PAGE and immunoblotting for elimination of high lipid, high detergent, low protein, non-SSP fractions.

Immunoaffinity enrichment for SSPs was done by HPLC (Beckman Instruments, Inc./System Gold: Ultraffinity-EP columns, 0.5 ml or 5.0 ml column capacity). The columns were derivatized, according to the manufacturer's instructions. The β and δ IgG were combined on a single column or used sequentially on individual columns. Precleared, solubilized protein in 0.1M potassium phosphate, pH 7.0, was passed through the column. The unbound fraction was collected; the bound proteins were eluted with 1.0 M potassium phosphate, pH 2.7 containing 0.5M KCl. Unbound and bound fractions were concentrated with 3-kilodalton (kDa) cut-off Centriprep-3® concentrators (Amicon, Inc.) and analyzed by protein concentration determination, SDS-PAGE, Western blots and ELISA.

Gel filtration was done using HPLC (Beckman Instruments Inc.). Superdex 200 HiLoad 16/60 prep grade or Sephacryl 16/60 HR-100 (Amershar Pharmacia Biotech) were used according to the manufacturer's instruction with 0.05M sodium phosphate, pH 7.0 containing 0.15 M NaCl. Fractions were concentrated and analyzed as in previous stages. Combined putative SSP fractions were further separated by size exclusion on a Superdex 75 HR 10/30 column (Amersham Thaandcia Biotedi) according to the manufacturer's instructions (elution buffer and subsequent analyses as above).

Anion exchange chromatography on a DEAS Sephacryl column (Amersham Pharmacia Biotech) was done using 0.02M Tris hydroxymethyl aminomethane pH 8.0, with a continuous salt gradient to 1.0M NaCl. Bound and unbound fractions were analyzed as previously. Further separations could be done using manufacturer's suggested appropriate buffers at pH 7.0 and 5.0 with a salt gradient. Bound and unbound fractions were analyzed for the presence of SSPs.

### Example 1

Affinity chromatography of solubilized membrane proteins, using same-sex antibodies (types β and δ) as ligand, produced the predicted enrichment for sex-typical molecules (not shown). This partially purified material (60% non-SSP removed over and above the approximately 20% non-SSP material that is removed by clearance on a pre-immune serum column) was used to immunize opposite sex rabbits. The resulting serum was used in Western Blots to determine whether like-sized molecules observed in male and female samples on 1-D electrophoresis were sex specific or nonspecific. Further enrichment for SSPs was done by gel filtration and then by ion exchange chromatography. Repeatable, characteristic profiles were seen, on SDS-PAGE, for male and female SSPs, in higher (50-60 kDa and above) and lower (35 kDa and below) MW ranges respectively.

Antibodies raised against purified male and female fetal SSPs (SSABs α and γ respectively) as well as non-SSABs (β and δ) were used in Western blots of sperm membrane proteins. Bands of approximately the same sizes as those of some male and female fetal SSPs were detected in these blots by α and γ antisera respectively (see FIG. 1) where sperm membrane antigens reacted to anti-fetal antibodies are shown on a Western blot. Lane designations as follows: Antigen: sperm head (H) and tail and midpiece (T) membrane proteins. Antibodies: female anti-male (α), female anti-female (β), male anti-female (γ), male anti-male (δ). Arrows indicate bands of molecular weight comparable to fetal SSPs.

FIG. 1, as noted, illustrates sperm membrane antigens detected by anti-fetal antibodies. Procedures for preparation of this figure involve use of sperm membrane preparations which were electrophoresed in a 12% polyacrylamide gel a detected after immunoblotting with female anti-male (α), female anti-female (β), male anti-female (γ) or male anti-male (δ) antiserum. Sperm membranes were solubilized from either the head region (H) or the tail and midpiece regions (T). Molecular weight standards (STD) are in the outer lanes. Arrows indicate proteins with molecular weights comparable to those previously observed for fetal SSPs.

### Example 2

Sex-chromosome-specific molecules on the sperm plasma membrane could be further studied, and ultimately isolated. In this approach we raised antibodies to sperm plasma membrane surface molecules in female rabbits and male rabbits. We called these antisera type epsilon (ε) and type zeta (ζ) respectively. These two immunizations produce different antibody responses, i.e. produce antibodies that would recognize different antigens, (Y- and X chromosomal respectively).

Specifically, immunization of a female rabbit will produce a response to Y-chromosome specific sperm-surface prod (Y-SCSPs), whereas X-chromosome specific proteins (X-SCSPs) will not. While not wishing to be bound by any one hypothesis, a working hypothesis is that a female (in this Example rabbit's) immune system would perceive Y-SCSFs, which would be male-related, to be “non-self”, whereas the sperm X-SCSPs possibly destined to be involved in female sex-determination, would be recognized as “self”. The converse would apply to male immunization. The two types of immunization would therefore produce antisera that could be used as ligands in immunoaffinity experiments, to isolate Y-SCSPs and X-SCSPs respectively.

### Example 3

Anti-Y (also called epsilon) and anti-X (also called zeta) type antiseraobtained as described above were used to produce affinity columns. Sperm membrane preparations were passed over these column. It was predicted that this would result in differential being of the putative sex-chromosome specific molecules. Material that was bound and material that was not bound by the two different columns could then be studio for their content of proteins. Used in this way, the epsilon and zeta columns should produce different arrays of bound and unbound proteins.

In pilot studies, we ran total sperm membrane proteins against one column (e.g. epsilon). Bound proteins were then eluted off the first column and then bound and unbound material run against the other column (zeta). Other samples were run in the converse sequence. This allowed us to observe which proteins bound to a particular column specifically, e.g. bound to epsilon but not to zeta, i.e., putatively Y-specific. Our pilot studies on chromatography of sperm plasma membrane proteins on these columns has produced preliminary results which suggest that we are obtaining some enhancement of larger molecular weight (MW) molecules from the epsilon column and, lower MW molecules from the zeta column. Thus we not only see differences; the differences are also in the direction we would have predicted from results on fetal SSPS, since our male SSPs are in the higher molecular weight range and the female SSPs in the lower range (see FIG. 1). These results indicate that this approach is likely to succeed in isolating sperm SCSPs.

The most repeatable result so far observed in these experiments is the occurrence of a putative X-SCSP band of about 32 kDa (range, in 6 experiment, between 31.0-32.8; mean 32.2). This band is designated to be a putative X-SCSP by virtue of being seen in the sample of proteins that was bound to zeta column and unbound to the epsilon (FIG. 2, lane 1), and being absent in samples that were bound to the epsilon and unbound to the zeta columns (see FIG. 2, lane 2). In five of the six experiments it was attempted to isolate Y chromosome-specific antigens; three of the five demonstrated enrichment of proteins ranging from 50-80 kDa.

The calculated variance of the R_{f} values (motility of protein relative to the dye front) of the 6 approximately 32 kDa bands was less than the variances of the two MW standards nearest in size (26 kDa and 38 kDa), known to be the same protein in each experiment. This supports the conclusion the approximate 32 kDa molecule is the same protein in each of the six experiments.

The sex-chromosome-specific molecules are repeatably identified in the SDS-PAGE gels, and are extracted from gels for further study by two-dimensional electrophoresis and Western blotting to establish whether the single band seen on one-dimensional electrophoresis contains one or more sex-specific molecule. Individual sex-chromosome-specific molecules can be used to raise monospecific antibodies, and for amino acid sequencing to derive a nucleotide sequence for PCR work.

### Example 4

The sex-chromosome-specific molecules we have been repeatedly identified in SDS-PAGE gels have been extracted for further study in 2-dimensional electrophoresis and Western blotting. The results of these studies in both cattle and pigs are provided in Table 1. Samples to the 2-dimensional gels with Western blotting are provided in FIG. 3 (which illustrates a porcine X sperm sample), FIG. 4 (which illustrates a porcine Y sperm sample), FIG. 5 (which illustrates a bovine sperm X sample), and FIG. 6 (which illustrates a bovine sperm Y sample). As may be seen in each of the figures, the spot identifier numbers referred to in Table 1 are illustrated in each of the respective figures.

### Example 5

The antibodies produced as described herein will be used to develop a method for separating the two classed of sperm cells. An established method for separating cells by use of antibodies will be used, for example, as follows. Commercially available, microscopically small magnetic beads are coated with appropriate antibodies (Olsaker et al., 1993, Anim. Genet 24:311), in this case either male chromosome-specific or female chromosome-specific antibodies (or with secondary antibody e.g. goat anti-rabbit IgG). The beads, coated, for example with X chromosome specific antibody, will be placed in a suspension of the sperm-cells, in an appropriate receptacle such as for example, glass dish. Because the sex-chromosome-specific proteins are present on the cell surface, the X-sperm cells will then bind to the female specific antibody on the beads, while the Y-sperm will not. The beads are then pulled to the side of the dish, using a magnet. Sperm cells of the two classes are recovered as those adhering to the beads (Y) and those not (X). Agglutination of sperm cells may also be used. In such an approach, live, unsorted sperm may be suspended in a serum free in vitro culture medium and exposed to either Y or X chromosome specific antibodies (as desired). Following treatment the medium is filtered in a glass wool filter, and sperm in the filtrate is used to perform in vitro fertilization.

### DISCUSSION

Affinity chromatography of male and female protein preparations, using the same IgG ligands (β, δ or both), produced differential enrichment for molecules of different MWs in the two sexes. This provided a basis for purification of SSPs.

Anti-male and -female SSABs (α and γ) respectively demonstrated bands in sperm membrane protein Western blots of −50-60 kDa and − or <35 kDa (FIG. 1), corresponding to the sizes of male and female fetal membrane proteins seen in SDS-PAGE. This supported the possibility that SCSPs, detectable by SSABs α and γ, may be present on the surfaces of X and Y sperm.

Indeed, affinity columns using ε and ζ antisera as ligands enriched for different sperm proteins. The ζ column, putatively capable of binding X-SCSPs, consistently isolated (n=6 of 6 trials) a molecule of −32 kDa (mean MW=32.2). The R_{f}s of these 6 bands had a smaller variance than the 26 kDa and 38 kDa MW standards. This indicates that these 6-32 kDa bands could represent the same molecule, which is interpreted to be a putative X-SCSP protein. The data suggest that it is a cell surface molecule, since the ε and δ antisera were produced by injecting intact, live sperm; B cells would, in contrast to cytotoxic T-cells, be expected to preferentially respond to surface molecules. Immunocytochemically, the resulting antisera did indeed react preferentially with cell surface proteins (and the acrosome),

Although not wishing to be bound by any one theory, the presence of different SCSPs on the surfaces of X and Y sperm implies post-meiotic transcription and/or translation (PMT/T of these molecules, and that they do not cross inter-spermatid cytoplasmic bridges. There is now ample evidence that PMT/T occurs (e.g., Hendriksen et al., 1995), and evidently not all PMT/T molecules cross the cytoplasmic bridges (Zheng, Y. et al. 8th Intl Symposium on Spermatology, Montreal, Canada 1998, Abstract P1-31). If, as suggested above, there is a selective advantage for the existence of these molecules, there would also be a selective pressure for them not to cross the bridges. One mechanism which might ensure this is that molecules destined for the plasma membrane might be placed there immediately after synthesis (Caldwell, K. A., Proc. Nat Acad. Science, USA 1991; 88:2407-2411). Alteratively, SCSP transcripts may be stored in spermatids in a “translationally repressed state” (Steger, K. Anat. Embryol (Berl) 1999; 199:471-487), and released from this state in the appropriate sperm cell. Finally, the sex chromosomes are mainly heterochromatic throughout meiosis; it is possible that the relevant loci may be inactivated, and that transcription occurs only after the bridges are closed.

Halving illustrated and described the principles of the invention in a preferred embodiment, it should be appreciated by those skilled in the art that the invention can be modified in arrangement and detail without departure from such principles. We claim all modifications coming within the scope of the following claims.

All publications, patents and patent applications referred to herein, are herein incorporated by reference in their entirety to the same extent as if each individual publication, patent or patent application was specifically and individually indicated to be incorporated by reference in its entirety.

### FULL CITATIONS OF REFERENCES CITED

Adimoelja, F. X. A. 1987. Sephadex gel for sex preselection. In New Horizons in Sperm Cell Research (Ed. H. Mohri.), Gordon and Breach Science Publishers, New York. pp 491-499.

Ali, J. I., Eldridge, F. E., Koo, G. C. and Scanbacher, B. D. 1990. Enrichment of bovine X- and Y-chromosome-bearing sperm with monoclonal H-Y antibody-fluorescence-activated cell sorter. Archives of Andrology 24: 235-245.

Amann, R. P. 1989. Treatment of sperm to predetermine sex. Theriogenology 31: 49-60.

Ambrose, J. D., Rajamahendran, R., Yoshiki T. and Lee, C. Y. 1996. Anti-bull sperm monoclonal antibodies: I. Identification of major antigenic domains of bull sperm and manifestation of interspecies cross-reactivity. Journal of Andrology 17(5): 567-578.

Bolton, A. E. and Hunter, W. M. 1973. The labelling of proteins to high specific radioactivities by conjugation to a 1251-containing acylating agent. Biochem J. 133(3): 529-539.

Cartwright, E. J., Harrington, P. M., Cowin, A. and Sharpe, P. T. 1993. Separation of bovine X and Y sperm based on surface differences. Molecular Reproduction and Development 34: 323-328.

Castle, P. E., Whaley, K. J., Hoen, T. E., Moench, T. R. and Cone, R. A. 1997, Contraceptive effect of sperm-agglutinating monoclonal antibodies in rabbits. Biology of Reproduction 56(1): 153-159.

Cran, D. G., Johnson, I. A, Miller, N. G. A., Cochrane, D. and Polge, C. 1993. Production of bovine calves following separation of X- and Y-chromosome bearing sperm and in vitro fertilizaton Veterinary Record 132:40-41.

Ericsson, R. J., Langevin, C. N. and Nishio, M. 1973. Isolation of fractions rich in human Y sperm. Nature 246:421-424.

Fenner, G., Johnson, L. A., Hruschka, W. R. and Bolt, D. J. 1992. Two-dimensional electrophoresis and densitometric analysis or solubilized bovine sperm plasma membrane proteins detected by silver staining and radioiodination. Archives of Andrology 29: 21-32.

Fraker P. J. and Speck, J. C., Jr. 1978. Protein and cell membrane iodinations with a sparingly soluble chloroamide, 1,3,4,6-tetachloro-3a,6a-diphrenylglycoluril. Biochemical and Biophysical Research Communications 80(4): 849-857.

Glasky, M. S. and Reading, C. L. 1989. Stability of specific immunoglobulin secretion by EBV-transformed lymphoblastoid cells and human-murine heterohybridomas. Hybridoma 8(4): 377-389.

Gledhill, B. L. 1988. Selection and separation of X- and Y-chromosome-bearing mammalian sperm., Gamete Research. 20: 377-395

Goldberg, E. H., Boyse, E. A., Bennett, D, Scheid, M4. and Carswell, E. A. 1971. Serological demonstration of H-Y (male) antigen on mouse sperm. Nature (Lond.) 232:478-480.

Harvey, E. N. 1946. Can the sex of mammalian offspring be controlled ? Journal of Heredity 37: 71-73.

Hendriksen, P. J. M., Tieman, M., van der Lende, T. and Johnson, L. A. 1993. Binding of anti-H-Y monoclonal antibodies to separated X and Y chromosome bearing porcine and bovine sperm. Molecular Reproduction and Development 35: 189-196.

Hendriksen, P. J. M., Hoogerbrugge, J. W., A. P. N., Koken, M. H. M., Koeimakers, J. H. J., Oostra, B. A., Van Per Lende, T., Grootegoed, J. A. 1995. Postmeiotic Transcription of X and Y chromosomal genes during spermatogenesis in the mouse. Dev. Biol, 170:730-733.

Hendriksen, P. J. M., Welch, G. R., Grootegoed, J. A.; Van Der Lende, T. and Johnson, L. A. 1996. Comparison of detergent-solubilized membrane and soluble proteins from flow cytometrically sorted X- and Y-chromosome bearing porcine spermatozoa by high resolution 2-D electrophoresis. Molecular Reproduction and Development 45: 342-350.

Howes, E. A., Miller, N. G. A., Dolby, C., Hutchings, A., Butcher, G. W. and Jones, R. 1997. A search for sex-specific antigens on bovine spermatozoa using immunological and biochemical techniques to compare the protein profiles of X and Y chromosome-bearing sperm populations separated by fluorescence-activated cell sorting. Journal of Reproduction and Fertility 110: 195-204.

Hudson, L., Hay, F. C. Practical Immunology, 2nd Edition. Blackwell Science Publications. Oxford 1980.

Johnson, L. A., Flook, J. P. and Hawk, H. W. 1989. Sex preselection in rabbits: live births from X and Y sperm separated by DNA and cell sorting. Biological Reproduction 41:199-203.

Kaneko, S., Oshio, S., Kobayashi, T., Izuka, R. and Mohri, H 1984. Human X- and Y-bearing sperm differ in cell surface sialic acid content. Biochemical and Biophysical Research Communications 124: 950-955.

Klint, M., Sege, K., Curan, B., Ploen, L., Peterson, P. A. 1985. Solublization and enrichment of boar sperm membrane proteins. Gam. Res, 11:335-348.

Lawrence J. and Singer R. 1986. Intracellular localization of MRNAS for cytoskeletal proteins. Cell 45, 407-415

Lee C., Levin A. and Branton, D.1987, Copper staining: a five-minute protein stain for sodium dodecyl sulfate-polyacrylamide gels. Analytical Biochemistry 166 (2): 308-312.

Marchalonis, J. J., Cone R. E. and Santer V. 1971. Enzymic iodination. A probe for accessible surface proteins of normal and neoplastic lymphocytes. Biochem J. 124(5): 921-927.

Markwell, M. A. 1982. A new solid-state reagent to iodinate proteins. I. Conditions for the efficient labeling of antiserum Analytical Biochemistry 125(2): 427-432.

Morrell, J. M., Keeler, K. D., Noakes, P. E., Mackenzie, N. M. and Dresser, P. W. 1988. Sexing of sperm by flow cytometry. Veterinary Record 122: 322-324.

Olsaker, I., Horgensen, C. B., Hellemann, A. L., Thomsen, P. D. and Lie, O. 1993. A fast and highly sensitive method for detecting freemartinism in bovine twins using immunomagnetic beads and Y-specific PCR primers. Animal Genetics 24(4): 311-313.

Peter, A. T., Jones, P. P. and Robinson, J. P. 1993. Fractionation of bovine spermatozoa for sex selection: a rapid immunomagnetic technique to remove spermatozoa that contain the H-Y antigen. Theriogenology 40: 1177-1185.

Pinkel, D., Lake, S., Gledhill, B. L., van Dilla, M. A., Stephenson, D. and Watchmaker, G. 1982. High resolution DNA content measurements of mammalian sperm. Cytometry 3: 1-9.

Rieger, D., Grisart, B., Semple, E., Van Langendonekt A., Betteridge, K. J. and Dessy, F. 1995. Comparison of the effects of oviductal cell co-culture and oviductal cell-conditioned medium on the development and metabolic activity of cattle embryos. Journal of Reproduction and Fertility 105(1): 91-98.

Rohde, W., Porstmann, T. and Dorner, G. 1973. Migation of Y-bearing human spermatozoa in cervical mucus. Journal of Reproduction and Fertilization 33: 167-169.

Rothschild, L. 1960. X and Y spermatozoa. Nature (Lond.) 187: 253-254.

Sambrook, J., Fritsch, E. F., Maniatis, T. Molecular Cloning: A Laboratory Manual. 2nd edition. Vol. 3, pp. 18.60-18.75. Cold Spring Harbor Laboratory Press 1989.

Sarkar, S. 1984. Motility, expression of surface antigen, and X and Y human sperm separation in in vitro fertilization medium. Fertility and Sterility 42: 899-905.

Sarkar, S., Jolly, D. J., Friedmann, T. and Jones, O. W. 1984. Swimming behaviour of X and Y human sperm. Differentiation 27: 120 -125.

Sills, E. S., Kirman, I., Colombero, L. T., Hariprashad, J., Rosenwaks, Z. and Palermo, G. D. 1998. H-Y antigen expression patterns in human X- and Y-chromosome-bearing spermatozoa. American journal of Reproductive Immunology 40: 43-47.

Steeno, O., Adimoelja, A. Steeno, J. 1975. Separation of X and Y bearing human spermatozoa with the sephadex gel-filtration method. Andrologia 7:95.

Steger, K. 1999. Transcriptional and translational regulation of gene expression in haploid spermatids. Anat Embryol (Berl.) 199(6): 471-487.

Sumner, A. T. and Robinson, J. A. 1976. A difference in dry mass between the heads of X- and Y-bearing human spermatozoa. Journal of Reproduction and Fertilization 48: 915.

Tack, B. F., Dean, J., Eilat, D., Lorenz, P. E. and Schechter, A. N., 1980. Tritium labeling of proteins to high specific radioactivity by reduction methylation. Journal of Biological Chemistry 255(18): 8842-8847.

Tao, M. H. and Levy, R. 1993. Idiotype-granulocyte-macrophage colony-stimulating factor fusion protein as a vaccine for B-cell lymphoma. Nature 362(6422): 755-758.

Tijssen, P. and Kurstak, E. 1984. Highly efficient and simple methods for the preparation of peroxidase and active peroxidase-antibody conjugates for enzyme immunoassays. Analytical Biochemistry 136(2): 451-457.

Utsumi, K. and Iritani, A. 1993. Embryo sexing by male specific antibody and by PCR using male specific (SRY) primer. Molecular Reproduction and Development 36(2): 238-241.

Wachtel S., Nakamura D., Wachtel, G., Felton, F., Kent, M. and Jaswaney, V. 1988. Sex selection with monoclonal H-Y antibody. Fertility and Sterility 50:355-360.

Windsor, D. P., Evans, G. and White, I. G. 1993. Sex predetermination by separation of X and Y chromosome-bearing sperm: A review. Reproduction Fertility and Development 5: 155-171.

Zheng, Y., Deng, X. and Martin-DeLeon, P. A. Compartmentalization of SPAM1 (PH-20) RNA in spermatids leading to transmission ratio distortion (TRD). 8^{th} International Symposium on Spermatology, Montreal, Canada, 1998, Abstract P1-31.

TABLE 1

| Spot Identifier | -MW (kD) | ˜pI Range |
|---|---|---|
| Bovine Sperm “X” Gel: | | |
| BXC6 | 24 | 5-5.5 |
| BXC7a,b | 23, 24 | 4.8-5.3 |
| BXC8 | 21 | 5.3-5.8 |
| BXC9 | 20 | 5.3-5.8 |
| BXC10 | 20 | 5.3-5.8 |
| BXC11 | 14 | 4.8-5.3 |
| BXC12 | 15 | 5-5.5 |
| Bovine Sperm “Y” Gel: | | |
| BYC18,19,20 | 27 | 5-6.5 |
| BYC21 | 20 | 5-5.5 |
| BYC27 | 9 | 5-5.6 |
| BYC28 | 9 | 5.3-5.8 |
| BYC29 | 5 | 5.3-5.8 |
| Porcine Sperm “X” Gel: | | |
| PXC1a,b | 99,100 | 5.3-5.7 |
| PXC5 | 43 | 5.3-5.7 |
| PXC6 | 53 | 6.1-6.7 |
| PXC7 | 31 | 5-5.6 |
| PXC8 | 30 | 6-6.5 |
| PXC11 | 25 | 7.5-9 |
| Porcine Sperm “Y” Gel: | | |
| PYC14a,b | 36,37 | 6.2-6.8 |

We claim:

1. A purified and isolated sex-chromosome-specific protein characterized as follows: (a) X chromosome specific; (b) associated with the cell membrane of bovine sperm cells; and (c) having a molecular weight on SDS-PAGE of about 32 kDa.

2. A purified and isolated sex-chromosome-specific protein having the characteristics of:
(a) X-chromosome-specific;
(b) associated with the cell membrane of a bovine sperm cell; and
(c) having a molecular weight on SDS-PAGE and pI range selected from the group consisting of 24, 5-5.5; 23, 4.8-5.3; 21, 5.3-5.8; 20, 5.3-5.8; 14,4.8-5.3; and 15, 555.

3. A purified and isolated sex-chromosome-specific protein having the characteristics of:
(a) Y-chromosome-specific;
(b) associated with the cell membrane of a bovine sperm cell; and
(c) having a molecular weight on SDS-PAGE and pI range selected from the group consisting of: 27, 5-6.5; 20, 5-5.5; 9, 5-5.6; 9, 5.3-5.8; and 5, 5.3-5.8.

4. A purified and isolated sex-chromosome-specific protein characterized as follows: (a) X chromosome specific, (b) associated with the cell membrane of porcine sperm cells; and (c) having a molecular weight and pI range selected on the group consisting of 99-100, 5.3-5.7; 43, 5.3-5,7; 53, 6.1-6.7, 31, 5-5.6; 30, 6-6.5; and 25, 7.5-9.

5. A purified and isolated sex-chromosome-specific protein characterized as follows: (a) Y chromosome specific; (b) associated with the cell membrane of porcine sperm cells; and (c) having a molecular weight and pI range selected on the group consisting of 36-37 and 6.2-6.8; respectively.

6. An isolated and purified nucleic acid sequence encoding a molecule according to any one of claims 1, 2, 3, 4 or 5.

7. A method for identifying sex-chromosome-specific molecules associated with animal sperm cell membranes, comprising:
(a) injecting whole sperm from a SP1 animal into a second and a third SP2 animal where the second and third animals are one of each of a male and female;
(b) harvesting antibodies raised in the second and third SP2 animals;
(c) separately reacting the antibodies from the second and third SP2 animal with a sperm cell membrane fraction from a SP1 animal;
(d) separating material in the cell membrane fraction which does not bind to the antibodies from the antibodies and bound material for each of the antibodies from the second and third SP2 animals;
(e) separating the bound material from the antibodies to create bound and unbound subfractions;
(f) comparing the bound material from the second SP2 animal antibodies with the bound material from the third SP2 animal antibodies and identifying as sex-chromosome-specific molecules bound material of one of the second and third SP2 animal antibodies not present in the other animal antibody bound material; and
(g) isolating the sex-chromosome specific molecules.

8. A method for identifying sex chromosome-specific molecules associated with animal sperm cell membranes, comprising:
(a) injecting a sperm cell fraction from a SP1 animal into a second and third SP2 animals;
(b) harvesting antibodies raised in the second and third SP2 animals;
(c) separately reacting the antibodies from the second and third SP2 with a sperm cell membrane fraction from a SP1 animal;
(d) separating material in the cell membrane fraction which does not bind to the antibodies from the antibodies and bound material for each of the antibodies from the second and third SP2 animals;
(e) separating the bound material from the antibodies to create bound and unbound subfractions;
(f) comparing the bound material from the second SP2 animal antibodies with the bound material from the third SP2 animal antibodies and identifying as sex-chromosome-specific molecules bound material of one of the second and third SP2 animal antibodies not present in the other antibody bound material; and
(g) isolating the sex-chromosome-specific molecules.

9. A method as claimed in claim 7 or 8 wherein the cell membrane fraction is obtained from a cell membrane of bovine or porcine sperm cells.

10. A method as claimed in any one of claim 7 or 8 wherein the cell membrane fraction is a plasma membrane, acrosomal membrane, mitochondrial membrane or endoplasmic reticulum membrane fraction.

11. A method as claimed in claim 10 wherein an anti-X chromosome specific molecule antibody is produced by immunizing male animals of a first animal species with sperm cell membrane fractions obtained from sperm cells of a second animal species.

12. A method as claimed in claim 10 wherein an anti-Y chromosome specific antibody is produced by immunizing female animals of a first animal species with sperm cell membrane fractions obtained from sperm cells of a second animal species.

13. A method as claimed in any one of claims 7 or 8, which further comprises preparing antibodies having specificity against an epitope of the sex-chromosome-specific molecule.

14. An antibody against an epitope of a sex-chromosome-specific molecule identified in accordance with the method as claimed in claim 13.

15. A method of screening for a sex-chromosome-specific molecule comprising reacting a test sample with an antibody as claimed in claim 14 and assaying for antigens in the test sample that bind to the antibody.

16. A method as claim in claim 15 wherein the antibody is labelled with a detectable substance.

17. A method for separating male and female determining sperm from native semen which comprises treating the native semen with one or more antibodies against an epitope of a sex-chromosome-specific molecule identified in accordance with the method as claimed in claim 13, to form conjugates between male or female determining sperm in the native semen and the antibodies, and isolating the conjugates, and sperm which have not bound to antibodies.

18. A kit for performing a method as claimed in claim 17 comprising antibodies against epitopes of sex-chromosome-specific molecules, and suitable supports in performing the methods of the invention.

19. A contraceptive comprising (a) a sex-chromosome-specific mol identified in accordance with a method as claimed claim 7 or 8 conjugated with a cytoxin which inactivates sperm; or (b) an antibody as claimed in claim 14.

20. A purified and isolated sex-chromosome-specific protein characterized as follows: (a) X chromosome specific; (b) associated with the cell membrane of bovine sperm cells.

21. A purified and isolated sex-chromosome-specific protein characterized as follows: (a) X chromosome specific; (b) associated with the cell membrane of bovine sperm cells; and (c) having a molecular weight on SDS-PAGE of about 6 kDa to about 40 kDa.

22. A purified and isolated sex-chromosome-specific protein characterized as follows: (a) Y chromosome specific; (b) associated with the cell membrane of bovine sperm cells.

23. A purified and isolated sex-chromosome-specific protein characterized as follows: (a) Y chromosome specific; (b) associated with the cell membrane of bovine sperm cells; and (c) having a molecular weight on SDS-PAGE of about 5 kDa to about 50 kDa.

24. The protein of claim 23 having a molecular weight range of about 10 kDa to about 25 kDa.

25. A purified and isolated sex-chromosome-specific protein characterized as follows: (a) X chromosome specific; (b) associated with the cell membrane of porcine sperm cells.

26. A purified and isolated sex-chromosome-specific protein characterized as follows: (a) X chromosome specific; (b) associated with the cell membrane of porcine sperm cells; and (c) having a molecular weight on SDS-PAGE of about 20 kDa to about 100 kDa.

27. A purified and isolated sex-chromosome-specific protein characterized as follows: (a) Y chromosome specific; (b) associated with the cell membrane of porcine sperm cells.

28. A purified and isolated sex-chromosome-specific protein characterized as follows: (a) Y chromosome specific; (b) associated with the cell membrane of porcine sperm cells; and (c) having a molecular weight on SDS-PAGE of about 5 kDa to about 50 kDa.

29. A molecule prepared according to a method of any one of claims 7 or 8.
